(19) United States
(12) Patent Application Publication
(10) Pub. No.: US 2026/0169009 A1
(43) Pub. Date: Jun. 18, 2026

(54) METHODS RELATED TO BIOLOGICAL AGE USING PROTEIN BIOMARKERS

(71) Applicant: Société des Produits Nestlé S.A., vevey (CH)

(72) Inventors: Rondo P MIDDLETON, CREVE COEUR, MO (US); Yuanlong Pan, Chesterfield, MO (US); James HOLZWARTH, Yverdon les Bains (CH); Ornella COMINETTI ALLENDE, Denges (CH); Berenice Bastien, St Louis, MO (US); Chang Woon Jang, Chesterbrook, PA (US)

(73) Assignee: Société des Produits Nestlé S.A., vevey (CH)

(21) Appl. No.: 19/319,320

(22) Filed: Sep. 4, 2025

Related U.S. Application Data
(60) Provisional application No. 63/733,714, filed on Dec. 13, 2024.

(51) Int. Cl. G01N 33/68

(52) U.S. Cl. CPC G01N 33/6893; G01N 2800/60; G01N 2800/7042

(57) ABSTRACT

The present invention comprises a method for determining the biological age of an animal, wherein the method comprises determining the level of at least one biomarker in said animal, and wherein the at least 1 biomarker is selected from the biomarkers as listed in Table 1.

### CROSS REFERENCE TO RELATED APPLICATION

This application claims priority to U.S. Provisional Application Ser. No. 63/733,714 filed Dec. 13, 2024 the disclosure of which is incorporated in its entirety herein by this reference.

### FIELD OF THE INVENTION

The present invention relates to a method for determining the biological age of a subject using levels of biomarkers in said subject. In particular, the present invention relates to a method for determining the biological age of a dog using levels of protein biomarkers in the dog. The invention further relates to methods of selecting a dietary, pharmacological or lifestyle regime for a subject, for example to provide a suitable dietary, pharmacological or lifestyle regime appropriate to the subject's biological age.

### BACKGROUND TO THE INVENTION

The ability to determine information regarding the health of an animal is desirable to inform about the animal's general health and well-being.

Chronological age is known to be a major indicator of general health status, with increasing chronological age associated with reduced health. However, depending on genetics, nutrition, and lifestyles, individuals may age slower or faster than their chronological age. Chronological age may therefore not always reflect an individual's rate of aging or risk of reduced health. On the other hand, the biological age of an individual (based on e.g. clinical biochemistry and cell biology measures) can vary compared to others of the same chronological age. Methods for determining biological age may be helpful for identifying individuals at risk of age-related disorders earlier than would be expected based on their chronological age.

However, there is a need for further methods of determining the biological age of an animal and utilising measures of biological age to improve health outcomes for an animal.

### SUMMARY OF THE INVENTION

In a first aspect, the present invention provides a method of determining the biological age of an animal subject, wherein the method comprises determining the levels of at least one biomarker in said animal, wherein the at least 1 biomarker is selected from the biomarkers as listed in Table 1.

Suitably, the method comprises determining the levels of five or more biomarkers in said animal, wherein at least 1 biomarker is selected from the biomarkers as listed in Table 1.

The present invention provides markers that allow the biological age of an animal subject to be determined. Advantageously, the determination of the biological age of an animal subject may be used to select a suitable dietary, pharmacological or lifestyle regime for the subject.

In one aspect, the present invention provides a method for selecting a dietary, pharmacological or lifestyle regime for an animal subject based on the determined biological age of the subject.

As used herein, ‘selecting a dietary, pharmacological or lifestyle regime for an animal subject’ may also encompass ‘recommending a dietary, pharmacological or lifestyle regime for the animal subject’ or ‘providing a recommended dietary, pharmacological or lifestyle regime for the animal subject’.

In a further aspect, the present invention provides a method for determining the efficacy of a dietary, pharmacological or lifestyle regime for improving the biological age of an animal subject, wherein the biological age of the subject is determined from a sample obtained from the subject before the dietary, pharmacological or lifestyle regime, the biological age of the subject is determined from a samples obtained from the subject after the dietary, pharmacological or lifestyle regime, and it is determined if there has been a change in the biological age of the subject before and after the dietary, pharmacological or lifestyle regime has been applied.

In a further aspect, the present invention provides a method for determining a likelihood that an animal subject will benefit from a dietary, pharmacological or lifestyle regime for improving the biological age of an animal subject, wherein the biological age of the subject is determined, and the subject is identified as being likely to benefit from a dietary, pharmacological or lifestyle regime for improving the biological age of an animal subject if it has an increased biological age relative to its chronological age or it has an biological age equal to its chronological age.

In a further aspect, the present invention provides a method of treating, preventing or reducing the risk of an age-related disease in an animal subject, wherein the method comprises determining the biological age of the subject, and administering a dietary, pharmacological or lifestyle regime to the subject.

For example, the age-related disease may be osteoarthritis, dementia, cognitive dysfunction, pre-diabetic condition, diabetes, cancer, heart disease, obesity, gastrointestinal disorders, incontinence, kidney disease, sarcopenia, frailty, vision loss, hearing loss, osteoporosis, cataracts, cerebrovascular disease, liver disease, and/or an immune system or immune-related disease or disorder.

In one aspect, the present invention provides a method of reducing the risk of an age-related disease in an animal subject, wherein the method comprises determining the biological age of the subject, and administering a dietary, pharmacological or lifestyle regime to the subject.

In one aspect, the present invention provides a dietary or pharmaceutical product for use in treating, preventing or reducing the risk of an age-related disease in an animal subject, wherein the dietary or pharmaceutical product is administered to a subject for which a biological age has been determined.

In one aspect, the present invention provides a dietary or pharmaceutical product for use in reducing the risk of an age-related disease in an animal subject, wherein the dietary or pharmaceutical product is administered to a subject for which a biological age has been determined.

In one aspect, the present invention provides the use of a dietary or pharmaceutical product for the manufacture of a medicament for the treatment, prevention or reduction of the risk of an age-related disease in an animal subject, wherein the medicament is administered to a subject for which a biological age has been determined.

In one aspect, the present invention provides the use of a dietary or pharmaceutical product for the manufacture of a medicament for the reduction of the risk of an age-related disease in an animal subject, wherein the medicament is administered to a subject for which a biological age has been determined.

In some embodiments, the method of treating, preventing or reducing the risk of an age-related disease is applied to a subject determined to be at risk of suffering from an age-related disease. In some embodiments, the dietary or pharmaceutical product for use in treating, preventing or reducing the risk of an age-related disease is administered to a subject determined to be at risk of suffering from an age-related disease.

In some embodiments, the risk of an animal subject suffering from an age-related disease is identified based on the determined biological age. In some embodiments, the subject may be considered at risk of suffering from an age-related disease where the subject is determined to have an increased biological age relative to its chronological age. In some embodiments, the subject may be considered at risk of suffering from an age-related disease where the subject is determined to have a biological age equal to its chronological age. In some embodiments, the subject may be considered at risk of suffering from an age-related disease wherein the subject is determined to be in a particular biological age category.

Suitably, the present methods comprise providing the levels of biomarkers in an animal subject from a sample obtained from the subject.

Suitably, the biomarkers are proteins.

The present invention further provides a computer-readable medium comprising instructions that when executed cause one or more processors to perform the method of the present invention.

The invention also provides a computer system for selecting a dietary, pharmacological or lifestyle regime for an animal subject, the computer system programmed to perform the steps of (i) determining the biological age of the subject, wherein the biological age is determined by the method of the present invention and (ii) selecting a suitable dietary, pharmacological, or lifestyle regime for the subject based on the determined biological age.

The invention also provides a computer system for determining the efficacy of a dietary, pharmacological or lifestyle regime for improving the biological age of an animal subject, the computer system programmed to perform the steps of (i) determining the biological age of the subject, wherein the biological age is determined by the method of the present invention from a sample obtained from the subject before administration of the dietary, pharmacological or lifestyle regime, (ii) determining the biological age of the subject, wherein the biological age is determined by the method of the present invention from a sample obtained from the subject after administration of the dietary, pharmacological or lifestyle regime, and (iii) comparing the determined biological ages from before and after administration of the dietary, pharmacological or lifestyle regime and determining if there has been a change in the biological age of the subject between the sample obtained from the subject before and after the dietary, pharmacological or lifestyle regime has been applied.

The invention also provides a computer system for determining a likelihood that an animal subject will benefit from a dietary, pharmacological or lifestyle regime for improving the biological age of an animal subject, the computer system programmed to perform the steps of (i) determining the biological age of the subject, wherein the biological age is determined by the method of the present invention and (ii) identifying a subject as likely to benefit from a dietary, pharmacological or lifestyle regime for improving the biological age of an animal subject if it has an increased biological age relative to its chronological age or if it has a biological age equal to its chronological age.

The invention also provides a computer system for selecting a dietary, pharmacological or lifestyle regime for an animal subject, the computer system programmed to perform the steps of (i) determining the biological age of the subject, wherein the biological age is determined by the method of the present invention, (ii) determining whether the subject is at risk of age-related disease, wherein the risk of age-related disease is determined based on the biological age of the subject and/or the comparison of the determined biological age and the chronological age, and (iii) selecting a suitable dietary, pharmacological, or lifestyle regime for the subject based on the determined risk of age-related disease.

The invention also provides a computer program product comprising computer implementable instructions for causing a programmable computer to (i) determine the biological age of an animal subject, wherein the biological age is determined by the method of the present invention, and (ii) select a suitable dietary, pharmacological, or lifestyle regime for the subject based on determined biological age and/or a comparison of the determined biological age and the chronological age.

The invention further provides a computer program product comprising computer implementable instructions for causing a programmable computer to (i) determine the biological age of an animal subject, wherein the biological age is determined by the method of the present invention, and wherein the biological age is determined from a sample obtained from the subject before a dietary, pharmacological or lifestyle regime has been applied to the subject, (ii) determine the biological age of the subject, wherein the biological age is determined by the method of the present invention, and wherein the biological age is determined from a sample obtained from the subject after a dietary, pharmacological or lifestyle regime has been applied to the subject, (iii) determine if there has been a change in the biological age of the subject between the sample obtained from the subject before and after the dietary, pharmacological or lifestyle regime has been applied, and (iv) determine the efficacy of the dietary, pharmacological or lifestyle regime in improving the biological age of the animal subject, wherein a reduction in the biological age of the subject indicates efficacy of the dietary, pharmacological or lifestyle regime.

### DESCRIPTION OF THE DRAWINGS

FIG. 1—Ages of animals in the dataset. Age given is the chronological age of the dog at sample collection. Dogs were split into groups based on their chronological age. A young group comprised 2-5 year-old dogs, and a senior group comprised 7+ year old dogs.

FIG. 2—An AdaBoost regressor was trained on a training dog proteomics dataset and optimal hyperparameters set. The AdaBoost regression model showed an r² value of 0.68 for the testing set with the optimal hyperparameters obtained from the grid search with 5-fold cross-validation (circles: training set, x's: testing set).

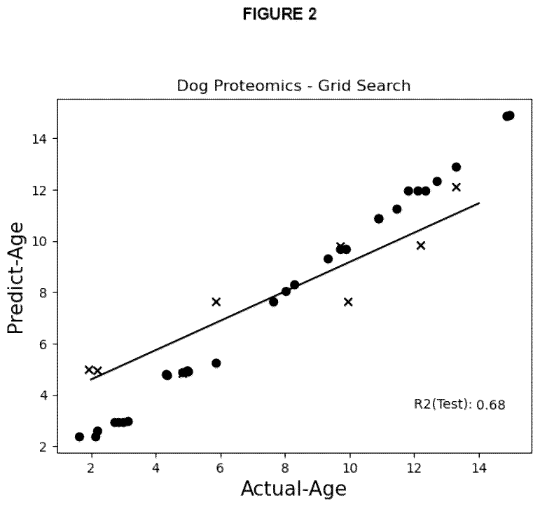

FIG. 3—Identification of the top 24 most important features in the AdaBoost Regressor model with their feature importance. Proteins are plotted in order of feature importance (bottom to top).

FIG. 4—Models were constructed using varying numbers of features. The r² value of the models is plotted against the number of features used in the model. The model using the top 24 most important features had an r² value of 0.82. The solid line indicates the reference r-squared value of 0.68 from the model containing all features.

FIG. 5—Ridge regression models were constructed with varying numbers of features. The r-squared (r²) value is plotted against the number of features used in the model. The ridge regression model reaches an r² value of 0.61 with top 24 important features. The solid line indicates the reference r-squared value of 0.68 from the Adaboost regression model containing all features.

FIG. 6—SVM classification plot for dog proteomics. It shows 100% accuracy to classify young and senior dogs.

FIG. 7—The categorical model accuracy was plotted as a function of the number of important proteins used in the model. The accuracy of the model reaches 0.8 when using only the top 13 most important features The model first reaches 100% accuracy with 94 proteins.

### DETAILED DESCRIPTION

### Subject

The present methods are directed to animal subjects. Suitably, the animal subject of the present invention is a mammal. In some embodiments of the invention, the subject is a canine. In some embodiments of the invention, the subject is a dog, suitably a domestic dog.

The present methods may utilise information regarding the breed of the dog. The dog may be categorised as a toy, small, medium, large or giant breed—for example. Suitably, the dog breed may be categorised based on the weight of the dog. Suitably, the dog breed may be categorised based on the average weight of a dog for a given breed.

Suitably, the dog may be categorised as a toy breed, small or medium breed, or large breed, or giant breed. Suitably, the categorisation is determined by the average weight of adult dogs of this breed. Suitably, a breed with an average weight between 2.2 kg and 5.5 kg is categorised as a toy breed, a breed with an average weight between 5.5 kg and 10 kg is categorised as a small breed and/or a breed with an average weight between 10 kg and 25 kg is categorised as a medium breed. A breed with an average weight between 26 kg and 45 kg is categorised as large breed and A breed with an average weight above 45 kg is categorised as giant breed.

Suitably, the sex of the animal may be classified as male or female.

### Chronological Age

Chronological age may be defined as the amount of time that has passed from the subject's birth to the given date. Chronological age may be expressed in terms of years, months, days, etc.

Suitably, the present method may be applied to a subject of any chronological age. In certain embodiments, the animal subject is a dog wherein the dog may be at least about 2 years old. Suitably, the dog may be at least about 2, at least about 3, at least about 4, at least about 5, at least about 6, at least about 7, at least about 8, at least about 9 or at least about 10 years old or older.

### Biological Age

Depending on genetics, nutrition, and lifestyles—for exampleindividuals may age slower or faster than their chronological age. Chronological age may therefore not always reflect an individual's biological rate of aging. As such, the biological age of an individual (based on e.g. clinical biochemistry and cell biology measures) can vary compared to others of the same chronological age.

The biological age of an individual is representative of the functional status of the cells and tissues of the individual, and how much an individual's body has been affected by ageing processes. The biological age of an individual can be useful to predict a risk of age-related diseases or conditions, as well as mortality risk.

The methods of the present invention determine the biological age of an animal. In some embodiments, the methods of the present invention determine the numerical biological age of an animal. Suitably, the numerical biological age is a continuous variable. The numerical biological age of an animal may be determined in terms of any suitable unit of time. Suitably, the numerical biological age of an animal is determined in terms of years or months. In some embodiments, the methods of the present invention determine a categorical biological age of an animal. Suitably, the categorical biological age is determined in terms of age categories based on age ranges of any suitable unit of time. Suitably, biological age categories may include numerical ranges in terms of years or months. In some embodiments, the categorical biological age is determined between two or more categories. Suitably, the categories of biological age may include categories such as junior, senior, infant, adult, geriatric, or mature. In some embodiments, wherein the animal subject is a domestic dog, the categories of biological age may include puppy, junior, adult, senior or mature. In some embodiments, wherein the animal subject is a domestic dog, the categories of biological age comprise junior, adult and senior. In some embodiments, wherein the animal subject is a domestic dog, the categories of biological age comprise junior and senior. Suitably, wherein the animal subject is a domestic dog, the categories of biological age are junior and senior, wherein junior includes biological ages of 2 years to less than 7 years, and senior includes biological ages of 7+ years.

Suitably, the methods of the present invention may be applied to an animal subject of any biological or chronological age.

### Sample

The present invention may comprise a step of determining the level of one or more biomarker from one or more sample obtained from an animal subject.

The present invention may comprise a step of determining the levels of at least one, preferably five or more, biomarkers from one or more sample obtained from an animal subject. In some embodiments, the levels of biomarkers are determined from a single sample obtained from an animal subject.

Suitably, the one or more sample may be a sample of any suitable biological material obtained from an animal subject. Suitably, the one or more sample may be a blood, urine, faecal, saliva, hair follicle, buccal swab, or tissue sample.

Suitably, the sample is derived from blood. The sample may contain a blood fraction or may be whole blood. In some embodiments, the sample is a serum sample. In some embodiments, the sample is a plasma sample.

### Biomarkers

The present invention comprises determining the level of biomarkers in an animal subject. Suitably, the biomarkers are proteins.

Suitably, the methods of the present invention comprise determining the level of one or more protein in an animal. Suitably, the level of one or more protein in an animal is determined from a sample obtained from said animal.

Suitably, the methods of the present invention comprise determining the level of one or more protein in the serum of an animal. Suitably, the level of one of more protein in the serum of an animal is determined from a serum sample obtained from said animal. Suitably, the methods of the present invention comprise determining the level of one or more protein in the plasma of an animal. Suitably, the level of one of more protein in the plasma of an animal is determined from a plasma sample obtained from said animal.

Determining the level of a protein in an animal may be carried out using any suitable method for determining protein levels known in the art.

### Proteins

A protein may be a polypeptide molecule formed from amino acids linked by peptide bonds. Suitably, the proteins may be specified using a protein name or gene name, wherein the gene name relates to the gene that encodes for the protein. Both the protein and gene names would be well known to, and understood by, the person of skill in the art. In some embodiments, the proteins are mammalian proteins, particularly canine proteins, for example domestic dog proteins.

Suitably, the level of a protein in a subject is given by the abundance of a protein quantified in a sample. Reference to a protein may be understood as a reference to all forms of the protein and to fragments and variants thereof. The abundance of a protein may include the abundance of a full-length protein, protein isoforms, secreted forms of proteins, and/or protein fragments.

Suitably, the levels of some or all of the proteins in Table 1, Table 2 and/or Table 3 are determined by any method of protein quantification known in the art. Suitable, exemplary methods are described below.

### Immunoassays

Levels of specific proteins can be determined from samples using immunoassays. These techniques may rely on antibody-based reagents that recognise and bind to specific proteins in a sample. Western Blotting involves 2-dimensional gel electrophoresis to separate proteins in a sample and transfer to a membrane, before detection of specific protein bands through antibodies linked to reporter molecules such as fluorescent moieties or enzymes that produce detectable moieties (such as horseradish peroxidase—HRP). Quantification can be accomplished through determination of the magnitude of antibody binding. Enzyme Linked Immunosorbent Assay (ELISA) also allows detection of a target protein through use of antibodies that are fluorophore- or enzyme-linked.

### Mass-Spectrometry Techniques

Simultaneous quantification of a large number of proteins can be achieved via mass spectrometry techniques. Bottom-up proteomic techniques comprise enzymatic digestion of the proteins present in a sample, and mass spectrometric identification and quantification of peptide fragments leading to quantification of protein abundances in biological samples such as serum (Geyer et al., 2016. Cell Syst. 2 (3): 185-95).

### Aptamer-Based Techniques

Quantification of proteins in samples obtained from a subject can be carried out using aptamer-based techniques such as SOMAscan®. Such methods utilise panels of aptamer molecules constructed from chemically modified nucleotides that form single-stranded DNA molecules that are capable of specific binding to proteins (Davies et al., 2012. Proc Natl Acad Sci USA. 109 (49): 19971-6). The levels of aptamer molecules that are bound to their corresponding proteins in a sample can be quantified by any standard DNA quantification method, such as microarrays or quantitative PCR (qPCR). The quantity of bound aptamer is relative to the quantity of a given protein in the sample, without requiring direct quantification of protein levels.

Suitably, an aptamer panel may be designed for use to detect and quantify proteins of a particular organism. In some embodiments, an aptamer panel designed for use in detecting and quantifying proteins of one organism may be used to detect and quantify proteins of a different organism. Suitably, said different organism is related to the organism whose proteins the panel is designed for use with. Suitably, both organisms are mammals. For example, an aptamer panel designed for human proteins may be used to detect and quantify canine proteins, in particular domestic dog proteins.

### Further Antibody-Based Techniques

Quantification or proteins in samples obtained from a subject can be carried out using antibody-based techniques such as Luminex®, Simoa® and Olink®. These techniques utilise protein-specific labelled antibodies. Antibody binding is converted into a unique quantifiable signal for each bound protein such as unique fluorescence or a DNA sequence detectable by qPCR.

### Methods

The methods of the present invention comprise determining the levels of at least one biomarker in an animal, wherein the at least 1 biomarker is selected from the biomarkers as listed in Table 1.

Suitably, the levels of at least 5, at least 6, at least 7, at least 8, at least 10, at least 11, at least 12, at least 13, at least 14, at least 15, at least 16, at least 17, at least 18, at least 19, at least 20, at least 21, at least 22, at least 23, at least 24, at least 25, at least 30, at least 40, at least 50, at least 75, or at least 100 biomarkers are determined, wherein at least 1 biomarker is selected from the biomarkers as listed in Table 1.

In some embodiments, the levels of at least 5, at least 10, at least 15, at least 24 or at least 50 biomarkers are determined, wherein at least 1 biomarker is selected from the biomarkers as listed in Table 1.

Suitably, the levels of five or more biomarkers in an animal are determined, wherein at least 2, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, or at least 100 biomarkers are selected from the biomarkers as listed in Table 1. Suitably, at least 2, at least 3, at least 4, at least 5, at least 10, at least 25, at least 50, at least 75 or at least 100 biomarkers are selected from the biomarkers as listed in Table 1.

In some embodiments, the levels of ten or more biomarkers in an animal are determined, wherein at least 2, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, or at least 100 biomarkers are selected from the biomarkers as listed in Table 1. In some embodiments, at least 2, at least 3, at least 4, at least 5, at least 10, at least 25, at least 50, at least 75 or at least 100 biomarkers are selected from the biomarkers as listed in Table 1.

In some embodiments, the levels of fifteen or more biomarkers in an animal are determined, wherein at least 2, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, or at least 100 biomarkers are selected from the biomarkers as listed in Table 1. In some embodiments, at least 2, at least 3, at least 4, at least 5, at least 10, at least 25, at least 50, at least 75 or at least 100 biomarkers are selected from the biomarkers as listed in Table 1.

In some embodiments, the levels of twenty-four or more biomarkers in an animal are determined, wherein at least 2, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, or at least 100 biomarkers are selected from the biomarkers as listed in Table 1. In some embodiments, at least 2, at least 3, at least 4, at least 5, at least 10, at least 25, at least 50, at least 75 or at least 100 biomarkers are selected from the biomarkers as listed in Table 1.

In some embodiments, the levels of fifty or more biomarkers in an animal are determined, wherein at least 2, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, or at least 100 biomarkers are selected from the biomarkers as listed in Table 1. In some embodiments, at least 2, at least 3, at least 4, at least 5, at least 10, at least 25, at least 50, at least 75 or at least 100 biomarkers are selected from the biomarkers as listed in Table 1.

In some embodiments, the levels of five or more biomarkers are determined, wherein at least 1, at least 2, at least 3, at least 4, at least 5, at least 6, at least 7, at least 8, at least 9, at least 10, at least 15, or at least 20 biomarkers are selected from the biomarkers as listed in Table 2. In some embodiments, the method comprises determining the levels of at least the biomarkers listed in Table 2. In some embodiments, the method comprises determining the levels of the biomarkers listed in Table 2.

In some embodiments, the levels of five or more biomarkers are determined, wherein at least 1, at least 2, at least 3, at least 4, at least 5, at least 6, at least 7, at least 8, or at least 9 biomarkers are selected from the biomarkers listed as biomarkers 1 to 10 in Table 2. In some embodiments, the method comprises determining the levels of at least the biomarkers listed as biomarkers 1 to 10 in Table 2. In some embodiments, the method comprises determining the levels of the biomarkers listed as biomarkers 1 to 10 in Table 2.

In some embodiments, the levels of at least 5 biomarkers are determined, wherein at least 5 of the biomarkers are selected from the biomarkers listed as biomarkers 1 to 10 in Table 2. In some embodiments, the at least 5 biomarkers comprise CEI, FGF9, AGAP2, SDSL, and GDF-11/8. In some embodiments, the at least 5 biomarkers comprise AMGO1, CARD9, SDSL, RUNX3, and FGF9. In some embodiments, the at least 5 biomarkers comprise FGF9, AGAP2, RUNX3, SDSL, and CF6. In some embodiments, the at least 5 biomarkers comprise CARD9, RN215, AMGO1, SDSL and CEI. In some embodiments, the at least 5 biomarkers comprise RUNX3, RN215, SDSL, FGF9, and CEI. In some embodiments, the at least 5 biomarkers comprise RN215, AMGO1, CF6, CEI, and SDSL. In some embodiments, the at least 5 biomarkers comprise CF6, AMGO1, FGF9, CEI, and RN215. In some embodiments, the at least 5 biomarkers comprise FGF9, CF6, CEI, CARD9, and GDF-11/8. In some embodiments, the at least 5 biomarkers comprise SDSL, RN215, AGAP2, CF6 and CEI.

In some embodiments, the levels of five or more biomarkers are determined, wherein at least 1, at least 2, at least 3 or at least 4 biomarkers are selected from the biomarkers listed as biomarkers 1 to 5 in Table 2. In some embodiments, the method comprises determining the levels of at least the biomarkers listed as biomarkers 1 to 5 in Table 2. In some embodiments, the method comprises determining the levels of the biomarkers listed as biomarkers 1 to 5 in Table 2.

In some embodiments, the levels of five or more biomarkers are determined, wherein at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80 or at least 90 biomarkers are selected from the biomarkers as listed in Table 3. In some embodiments, the method comprises determining the levels of at least the biomarkers listed in Table 3. In some embodiments, the method comprises determining the levels of the biomarkers listed in Table 3.

In some embodiments, the levels of five or more biomarkers are determined, wherein at least 1, at least 2, at least 3, at least 4, at least 5, at least 6, at least 7, at least 8, at least 9, at least 10, at least 15, at least 20, at least 25, at least 30, at least 35, at least 40 or at least 45 biomarkers are selected from the biomarkers listed as biomarkers 1 to 50 in Table 3. In some embodiments, the method comprises determining the levels of at least the biomarkers listed as biomarkers 1 to 50 in Table 3. In some embodiments, the method comprises determining the levels of the biomarkers listed as biomarkers 1 to 50 in Table 3.

In some embodiments, the levels of five or more biomarkers are determined, wherein at least 1, at least 2, at least 3, at least 4, at least 5, at least 6, at least 7, at least 8, at least 9, at least 10, at least 11, at least 12, at least 13 or at least 14 biomarkers are selected from the biomarkers listed as biomarkers 1 to 15 in Table 3. In some embodiments, the method comprises determining the levels of at least the biomarkers listed as biomarkers 1 to 15 in Table 3. In some embodiments, the method comprises determining the levels of the biomarkers listed as biomarkers 1 to 15 in Table 3.

In some embodiments, the levels of five or more biomarkers are determined, wherein at least 1, at least 2, at least 3, at least 4, at least 5, at least 6, at least 7, at least 8 or at least 9 biomarkers are selected from the biomarkers listed as biomarkers 1 to 10 in Table 3. In some embodiments, the method comprises determining the levels of at least the biomarkers listed as biomarkers 1 to 10 in Table 3. In some embodiments, the method comprises determining the levels of the biomarkers listed as biomarkers 1 to 10 in Table 3.

In some embodiments, the levels of at least 5 biomarkers are determined, wherein at least 5 of the biomarkers are selected from the biomarkers listed as biomarkers 1 to 10 in Table 3. In some embodiments, the at least 5 biomarkers comprise Laminin-2, NXF1, CEI, NTRI and ARHG1. In some embodiments, the at least 5 biomarkers comprise NTRI, RUNX3, NXF1, AGAP2 and ARHG1. In some embodiments, the at least 5 biomarkers comprise ARHG1, AGAP2, CEI, NXF1 and RUNX3. In some embodiments, the at least 5 biomarkers comprise ADAMTS-5, NTRI, IGDC4, CEI and NXF1. In some embodiments, the at least 5 biomarkers comprise CEI, RUNX3, NTRI, AGAP2 and ADAMTS-5. In some embodiments, the at least 5 biomarkers comprise NXF1, NTRI, IGDC4, RUNX3 and CEI. In some embodiments, the at least 5 biomarkers comprise RUNX3, NTRI, IGDC4, Laminin-2 and NXF1. In some embodiments, the at least 5 biomarkers comprise Laminin-2, CEI, ARHG1, IGDC4 and NTRI.

In some embodiments, the levels of five or more biomarkers are determined, wherein at least 1, at least 2, at least 3 or at least 4 biomarkers are selected from the biomarkers listed as biomarkers 1 to 5 in Table 3. In some embodiments, the method comprises determining the levels of at least the biomarkers listed as biomarkers 1 to 5 in Table 3. In some embodiments, the method comprises determining the levels of the biomarkers listed as biomarkers 1 to 5 in Table 3.

As used herein, GDF11/8 may refer to GDF11, GDF8, or each of GDF11 and GDF8.

### Further Biomarkers

In some embodiments, the methods of the present invention may comprise determining the levels of additional biomarkers in an animal that are not listed in Tables 1-3. The methods of the present invention may comprise determining the levels of additional biomarkers of any type. In some embodiments, the methods of the present invention may comprise determining the levels of additional biomarkers that are not proteins in an animal, further to at least 1 protein biomarker selected from the biomarkers as listed in Table 1.

Suitably, the additional biomarkers may be any type of biological molecule or physiological characteristic. Biomarkers according to the present invention may include, for example, proteins, mRNA, small molecules including but not limited to metabolites, genes (e.g. methylation), cells, and ions.

### Determination of Biomarkers Indicative of Biological Age of an Animal

The present invention comprises determining the levels of biomarkers in an animal and determining the biological age of an animal from said levels of biomarkers.

The provision of levels of biomarkers that are indicative of biological age may be achieved through training datasets and machine learning approaches, for example. Suitably, the machine learning approaches may be supervised machine learning approaches. Suitably, the model may be a multinomial regression, support-vector machine (SVM), a random forest or an Adaptive Boosting (AdaBoost) model.

By way of example, levels of biomarkers in individual animals may be trained against a dataset comprising animals of known chronological age. Suitably, levels of biomarkers may be trained against a dataset comprising animals that were known to subsequently develop age-related disease or die after the samples from which the dataset was generated were taken.

Suitably, the chronological age of an animal may be referred to as the age of an animal.

For example, models of biomarkers indicative of biological age may be provided by training a dataset of a range of biomarker levels in individuals against a training dataset of animals with a known age using a machine learning framework, and testing against a with-held cohort to validate the veracity of the model.

The machine learning framework may comprise fitting a multinomial model, a random forest, an AdaBoost, a SVM (support vector machine), penalized multinomial logistic regression or other model used to predict multi class outcomes.

Suitably, the machine learning framework may be used to determine a model comprising a set of biomarkers and biomarker levels that are indicative of biological age.

The model may comprise the levels of biomarkers; wherein the level of a biomarker is considered in the model by multiplying by a coefficient value.

The coefficient value for each parameter typically depends on the measurement units of all the variables in the model. As would be understood by the skilled person, the value for each coefficient value will therefore depend on, for example, the number and nature of the different parameters used in the model and the nature of the training data provided. Accordingly, routine statistical methods may be applied to a training data set in order to arrive at coefficient values.

Suitably, the machine learning platform may comprise one or more deep neural networks. Neural Networks are collections of neurons (also called units) connected in an acyclic graph. Neural Network models are often organized into distinct layers of neurons. For most neural networks, the most common layer type is the fully connected layer in which neurons between two adjacent layers are fully pairwise connected, but neurons within a single layer share no connections. One of the main features of deep neural networks is that neurons are controlled by non-linear activation functions. This non-linearity combined with the deep architecture make possible more complex combinations of the input features leading ultimately to a wider understanding of the relationships between them and as a result to a more reliable final output. Deep neural networks have been applied for many types of data ranging from structural data to chemical descriptors or transcriptomics data.

Suitably, the machine learning platform comprises one or generative adversarial networks. Suitably, the machine learning platform comprises an adversarial autoencoder architecture. Suitably, the machine learning platform comprises a feature importance analysis for ranking biomarkers by their importance in determining biological age.

### Comparison to a Reference or Control

The present method may comprise a step of comparing the difference in the level(s) of one or more biomarker in the test sample to one or more reference or controls. The level(s) of one or more biomarker in the reference or control may be associated with biological age. In some embodiments, the reference value is a value obtained previously for a subject or group of subjects with a known biological age. The reference value may be based on a known level(s) of one or more biomarker, e.g. a mean or median level, from a group of subjects with known biological age.

The reference level(s) of one or more biomarker may comprise level(s) of one or more biomarker from at least 1, at least 2, at least 4, at least 10, at least 20, at least 40, at least 80, at least 100, at least 150, at least 200, at least 300, at least 400 animals, at least 500, at least 750 or at least 1000 animals.

### Age-Associated Diseases

Biological age is a risk factor for a variety of diseases. In particular, a biological age greater than the chronological age of an animal may be predictive of a greater risk of disease. A biological age equal to the chronological age of an animal may also be predictive of a greater risk of disease.

As an example, dogs with greater biological ages are more likely to suffer from renal disease, immune system or immune-related diseases or disorders, cancer, cardiovascular diseases, neurodegenerative diseases and musculoskeletal diseases.

Suitably, the methods of the present invention may comprise determining the risk of an age-related disease. Suitably, the risk of an age-related disease may be determined from the determined biological age of an animal. The risk of an age-related disease may be determined from a determined numerical biological age, and/or from a determined categorical biological age. Suitably, the risk of an age-related disease may be determined from the comparison of the determined biological age of an animal with the chronological age of an animal. For example, where an animal is determined to have a biological age equal to or greater than its chronological age, the risk of an age-related disease may be increased. Where an animal is determined to have a biological age younger than its chronological age, the risk of an age-related disease may be reduced. Suitably, the determination of the risk of an age-related disease may comprise the determination of the biological age of an animal according to the present invention, combined with other suitable health indicators known in the art. For example, the determined biological age of an animal may be combined with other disease indicators or health metrics known in the art, such as an animal's weight, blood glucose, cholesterol or triglyceride levels, inflammation markers, oxidative stress markers, or results of a physical examination.

It is to be appreciated that all references herein to treatment include curative, palliative and prophylactic treatment, although in the context of the invention references to preventing are more commonly associated with prophylactic treatment. Treatment may also include arresting progression in the severity of a disease.

### Method for Selecting a Dietary, Pharmacological, or Lifestyle Regime

In a further aspect, the present invention provides a method for selecting a dietary, pharmacological, or lifestyle regime for a subject. Suitably, the method is for selecting a dietary, pharmaceutical, or lifestyle regimen for a dog.

Suitably, the method for selecting a dietary, pharmacological, or lifestyle regime for a subject comprises determining the biological age of the subject by methods according to the present invention. A dietary, pharmacological, or lifestyle regimen may be selected based on the determined biological age of the subject. In some embodiments, the selection of a dietary, pharmacological, or lifestyle regime for a subject is based on a determined numerical biological age of a subject. In some embodiments, the selection of a dietary, pharmacological, or lifestyle regime for a subject is based on a determined categorical biological age of a subject. In some embodiments, the selection of a dietary, pharmacological, or lifestyle regime for a subject is based on a comparison between the determined biological age of a subject and the subject's chronological age. Suitably, a dietary, pharmacological, or lifestyle regime may be selected for a subject wherein the determined biological age is equal to or greater than the subject's chronological age.

The dietary, pharmacological, or lifestyle regime may be applied to the dog for any suitable period of time. By way of example, the dietary, pharmacological, or lifestyle regime may be applied for at least 2, at least 4, at least 8, at least 16, at least 32, or at least 64 weeks. The dietary, pharmacological, or lifestyle regime may be applied for at least 3, at least 6, at least 12, at least 24, at least 36, at least 48 or at least 60 months. Suitably, the dietary, pharmacological, or lifestyle regime may be applied for at least 1, at least 2, at least 3, at least 4, at least 5, at least 6, at least 7, at least 8, at least 9 or at least 10 years. Suitably, the dietary, pharmacological, or lifestyle regime may be applied for the lifetime of the subject.

Suitably, the modification is a dietary intervention as described herein. By the term “dietary intervention” it is meant an external factor applied to a subject which causes a change in the subject's diet. More preferably the dietary intervention includes the administration of at least a dietary product or a dietary regimen or a nutritional or nutraceutical supplement.

The dietary regime may be a meal, a regime of meals, a supplement or a regime of supplements, or combinations of a meal and a supplement, or combinations of a meal and multiple supplements.

The dietary intervention or dietary product described herein may be any suitable dietary regime, for example, a calorie-restricted diet, a senior diet, a low protein diet for young animals or a high protein diet for senior animals, an anti-inflammatory diet, potassium supplement diet, polyunsaturated fatty acids (PUFA) supplement diet, anti-oxidant supplement diet, a vitamin B supplement diet, liquid diet, selenium supplement diet, omega 3-6 ratio diet, or diets supplemented with carnitine, branched chain amino acids or derivatives, nucleotides, nicotinamide precursors such as nicotinamide mononucleotide (MNM) or nicotinamide riboside (NR) or any combination of the above.

Suitably, the dietary intervention or dietary product may be a calorie-restricted diet, a high-calorie diet, a senior diet, or a low protein diet for young animals and a high protein for senior animals. Suitably, the dietary intervention or dietary product may be a calorie-restricted diet. Suitably, the dietary intervention or dietary product may be a low protein diet for young animals and a high protein diet for senior animals.

A dietary intervention may be determined based on the baseline maintenance energy requirement (MER) of the subject. Suitably, the MER may be the amount of food that stabilizes the dog's body weight (less than 5% change over three weeks). Suitably the MER may be predicted by the determined biological age of an animal.

By way of example, it is generally understood that younger, growing dogs benefit from a high energy/high protein diet; however, older dogs may have a lower energy requirement and therefore diets can be appropriately modified. In particular, many manufacturers produce a ‘senior’ range of dog food which is lower in calories, higher in fibre and protein and suitable levels of fat for an older dog.

Suitably, a calorie-restricted diet may comprise about 50%, about 55%, about 60%, about 65%, about 75%, about 80%, about 85%, or about 90% of the animal's MER. Suitably, a calorie-restricted diet may comprise about 60% or about 75% of the animal's MER.

Suitably, a low-protein diet may comprise less than 20% protein (% dry matter). For example, a low-protein diet may comprise less than 19% (% dry matter). A high protein diet may comprise more than 20% protein (% dry matter), for example a high protein diet may comprise more than 30% protein (% dry matter).

The dietary intervention may comprise a food, supplement and/or drink that comprises a nutrient and/or bioactive that mimics the benefits of caloric restriction (CR) without limiting daily caloric intake. For example, the food, supplement and/or drink may comprise a functional ingredient(s) having CR-like benefits. Suitably, the food, supplement and/or drink may comprise an autophagy inducer. Suitably, the food, supplement and/or drink may comprise fruit and/or nuts (or extracts thereof). Suitable examples include, but are not limited to, pomegranate, strawberries, blackberries, camu-camu, walnuts, chestnuts, pistachios, pecans. Suitably, the food, supplement and/or drink may comprise probiotics with or without fruit extracts or nut extracts.

By way of example, animals with a low biological age may be more active than animals with a high biological age. In some embodiments, the animal subject is a dog. Suitably, a low biological age may be a biological age of 2-6 years (i.e. a “young” dog as categorised in categorical biological age). A dog food composition having a ratio of energy from protein to energy from fat below 0.80 may be advantageous to dogs with a low biological age. A food composition high in protein and high in fat is particularly well adapted for dogs with a low biological age. Typically, a dog food composition for dogs with a low biological age has from about 20-30% protein and from about 15-25% fat. Indeed, a food composition dense in energy from fat will provide a dog with a low biological age with sufficient energy for the moderate to very intense activities (i.e., brisk walk to fast run) in which it spontaneously gets involved. Furthermore, the energy from protein to the energy from fat ratio is found to be advantageous in such a food composition for maintaining the lean body mass of dogs with a low biological age.

Similarly, animals with a high biological age may be less active than animals with a low biological age. In some embodiments, the animal subject is a dog. Suitably, a high biological age may be a biological age of 7+ years (i.e. a “senior” dog as categorised in categorical biological age). A particularly well adapted dog food composition for a dog with a high biological age may have the ratio of energy from protein to energy from fat in such a food composition greater than 0.80. More specifically, a protein content from about 20-30% or higher and less than about 15% fat. Because they may have a low resting metabolic rate, such a food composition is ideally adapted to dogs with a high biological age. The composition will have the effect of limiting the fat and/or carbohydrate intake of dogs with a high biological age and therefore their tendency to be overweight.

The modification in lifestyle may be any change as described herein, e.g. a change in exercise regime.

Similar to a dietary intervention, the determination of a biological age may allow a determination to switch the test subject to an appropriate exercise regime.

Ideal activity level and type may differ according to determined biological age. For example, wherein the animal subject is a dog, a dog with a high biological age may be spontaneously engaged in mild (e.g., slow walking), moderate (e.g., brisk walking) or occasionally intense (e.g., running) activity types. A dog with a low biological age, in comparison, may mainly be voluntarily involved in moderate, intense or very intense (e.g., fast running) activities.

The pharmacological regime may refer to administration of a therapeutic modality or regimen. The modality may be a modality useful in treating and/or preventing—for example—arthritis, dental diseases, endocrine disorders, heart disease, diabetes, liver disease, kidney disease, prostate disorders, cancer and behavioural or cognitive disorders.

Suitably, a therapeutic modality or regimen may include any suitable therapeutic agent. A therapeutic modality or regimen may include any therapy or therapeutic agent suitable to treat, prevent, diagnose or reduce the risk of a disease or condition. In some embodiments, a therapeutic modality or regimen may be suitable for improving the biological age of a subject. Suitably, a therapeutic modality or regimen may include any of small molecules, biologics, and gene therapies.

Suitably, prophylactic therapies may be administered to a subject identified as being at risk of such disorders due to the biological age of the subject. In other embodiments, subjects determined to be at risk of certain conditions due to biological age may be monitored more regularly so that diagnosis and treatment can begin as early as possible.

### Method for Determining the Efficacy of a Dietary, Pharmacological or Lifestyle Regime

In a further aspect, the present invention provides a method for determining the efficacy of a dietary, pharmacological, or lifestyle regime that is administered to an animal subject. Suitably, the present invention provides a method for determining the efficacy of a dietary, pharmaceutical, or lifestyle regime in improving the biological age of an animal subject. Suitably, the method is for determining the efficacy of a dietary, pharmaceutical, or lifestyle regime in improving the biological age of an animal subject wherein the biological age of the subject has been determined by the methods disclosed herein.

Suitably, a method for determining the efficacy of a dietary, pharmacological, or lifestyle regime administered to an animal subject comprises determining the biological age of the subject according to the methods of the present invention prior to the administration of the dietary, pharmacological, or lifestyle regime to the subject, determining the biological age of the subject according to the methods of the present invention after the administration of the dietary, pharmacological, or lifestyle regime to the subject, and determining if there has been a change in the biological age of the subject between prior to the administration and after the administration of the dietary, pharmacological, or lifestyle regime to the subject.

Suitably, at least one of the determination of the subject's biological age prior to the administration of the dietary, pharmacological, or lifestyle regime to the subject, or the determination of the subject's biological age after the administration of the dietary, pharmacological, or lifestyle regime to the subject may be achieved by a method of determining the biological age of a subject according to the present invention. Alternative methods of determining biological age of a subject may be used instead of, or in addition to, the methods according to the present invention, and further determinations of biological age of the subject prior to or after administration of the dietary, pharmacological, or lifestyle regime to the subject may be carried out, so long as at least one of the determinations of the biological age of the subject is carried out according to the methods of the present invention.

In some embodiments, the biological age of the subject may be determined multiple times prior to the administration of the dietary, pharmacological, or lifestyle regime to the subject. In some embodiments, the biological age of the subject may be determined multiple times after the administration of the dietary, pharmacological, or lifestyle regime to the subject.

Suitably, the determination of the biological age of the subject after administration of the dietary, pharmacological, or lifestyle regime to the subject may be carried out a suitable period after the administration of the dietary, pharmacological, or lifestyle regime to the subject. The period may be, for instance, at least 1 day, at least 2 days, at least 3 days, at least 4 days, at least 5 days, at least 6 days, at least 7 days, at least 14 days, at least 3 weeks, at least 4 weeks, at least 5 weeks, at least 6 weeks, at least 7 weeks, at least 8 weeks, at least 3 months, at least 4 months, at least 5 months, at least 6 months, or at least a year after administration of the dietary, pharmacological, or lifestyle regime to the subject.

In some embodiments, the method may comprise the selection of an alternative dietary, pharmacological, or lifestyle regime for administration to an animal subject, where the previous dietary, pharmacological, or lifestyle regime is determined to be inefficient in improving the biological age of the subject. In some embodiments, the method may comprise changing the administration frequency or dosage of a pharmacological regime that is determined to be inefficient in improving the biological age of the subject. In some embodiments, the method may comprise adjusting the dietary regime administered to the animal subject, wherein the dietary regime is determined to be inefficient in improving the biological age of the subject. Suitably the dietary regime may be adjusted to change the amount of food provided to the subject, the feeding time, and/or the frequency of feeding. In some embodiments, the method may comprise adjusting the lifestyle regime administered to the subject, wherein the lifestyle regime is determined to be inefficient in improving the biological age of the subject. Suitably, the lifestyle regime may be adjusted to change the amount, frequency and/or time of exercise of the subject.

### Method for Determining the Likelihood of Benefit of a Dietary, Pharmacological or Lifestyle Regime

In a further aspect, the present invention provides a method for determining the likelihood that an animal subject will benefit from a dietary, pharmacological, or lifestyle regime. Suitably, the dietary, pharmacological, or lifestyle regime is for improving the biological age of the animal subject.

Suitably, a method for determining the likelihood that an animal subject will benefit from a dietary, pharmacological, or lifestyle regime for improving the biological age of a subject comprises determining the biological age of the subject by a method described herein, and identifying the subject as being likely to benefit from a dietary, pharmacological, or lifestyle regime for improving the biological age of a subject based on the determined biological age. Suitably, a subject may be likely to benefit from a dietary, pharmaceutical, or lifestyle regime for improving biological age of a subject where the subject has a determined biological age that is higher than the chronological age of the subject. In some embodiments, a subject may be likely to benefit from a dietary, pharmaceutical, or lifestyle regime for improving biological age of a subject wherein the subject has a determined biological age that is high. Where the subject is a dog, for instance, a high biological age may be a determined biological age of at least 7 years, at least 8 years, at least 9, at least 10, at least 11, at least 12, at least 13, at least 14, or at least 15 years. Suitably, where the subject is a dog, a high biological age is a determined biological age of at least 7 years.

In some embodiments, a subject may be likely to benefit from a dietary, pharmaceutical, or lifestyle regime for improving biological age of a subject where the subject has a determined biological age that equal to or close to the chronological age of the subject. Suitably, where the biological age is determined in years, close to may refer to where the determined biological age is within 2 years, within 1 year, or within half a year of the chronological age of the subject. Suitably, where the biological age is determined in months, close to may refer to where the determined biological age is within 24 months, within 18 months, within 12 months, within 9 months, within 6 months, within 4 months, within 3 months, within 2 months or within 1 month of the chronological age of the subject. Suitably, where the biological age is determined in years, equal to may refer to where the determined biological age is the same age in years as the chronological age of the subject. Suitably, where the biological age is determined in months, equal to may refer to where the determined biological age is the same age in months as the chronological age of the subject.

The present invention may thus advantageously enable the identification of dogs that are expected to respond particularly well to a given intervention (e.g. dietary, pharmacological or lifestyle regime). The intervention can thus be applied in a more targeted manner to dogs that are expected to respond due to their determined biological age.

### Use of a Dietary Intervention or Pharmaceutical Product

In one aspect, the present invention provides a dietary or pharmacological product for use in improving the biological age of an animal subject, wherein the dietary or pharmaceutical product is administered to the subject. Suitably, the dietary or pharmaceutical product is administered to a subject for which a biological age has been determined by the methods described herein.

Suitably, an improvement in the biological age of an animal subject may be a reduction of the biological age of the subject. In some embodiments, an improvement in the biological age of an animal subject may be a reduction of the biological age of the subject relative to the chronological age of the subject. For instance, an improvement in the biological age of an animal subject may be a reduction of the biological age of the subject to match or be more similar to the chronological age of the subject. In some embodiments, an improvement in the biological age of an animal subject may be a reduction of the biological age of the subject to be lower than the chronological age of the subject.

In one aspect, the present invention provides a dietary or pharmacological product for use in reducing the risk of an age-related disease in an animal subject, wherein the dietary or pharmaceutical product is administered to the subject. Suitably, the dietary or pharmaceutical product is administered to a subject for which a biological age has been determined by the methods described herein. In one aspect, the dietary or pharmacological product is administered to an animal subject which has been determined to be at risk of an age-related disease by determination of the subject's biological age by the methods described herein.

### Ecosystem

Suitably, the present methods may comprise an ‘ecosystem’, in particular a digital ecosystem. Suitably, the present methods may comprise providing a sample obtained from the animal subject, optionally using a kit according to present invention; and (b) providing the sample (e.g. by mailing) for subsequent determination of the level of one or more biomarker(s) in the sample.

The level of one or more biomarker(s) may then be used according to any of the present methods; preferably using a computer system or a computer program product according to the present invention.

The computer system or computer program may then prepare and share a report detailing the outcome of analysis/method in the form of e.g. selecting or recommending a suitable lifestyle regime, dietary regime or therapeutic intervention for a dog or any other outcome of the present methods.

### Computer-Readable Medium and Computer System

The present methods may be performed using a computer. Accordingly, the present methods may be performed in silico.

Suitably, the computer may prepare and share a report detailing the outcome of the present methods.

The methods described herein may be implemented as a computer program running on general purpose hardware, such as one or more computer processors. In some embodiments, the functionality described herein may be implemented by a device such as a smartphone, a tablet terminal or a personal computer.

In one aspect, the present invention provides a computer program product comprising computer implementable instructions for causing a programmable computer to determine the biological age of an animal as described herein. Suitably, the computer program product comprises a computer-readable medium comprising said computer-implementable instructions.

In another aspect, the present invention provides a computer program product comprising computer implementable instructions for causing a device to determine the biological age of an animal subject; and optionally select a suitable dietary, pharmacological, or lifestyle regime for the subject based on the subject's biological age, as determined by the present method. The computer program product may also be given additional parameters or characteristics for the subject. Suitably, the computer program product comprises a computer-readable medium comprising said computer-implementable instructions.

In another aspect, the present invention provides a computer program product comprising computer implementable instructions for causing a device to determine whether an animal subject is at a risk of developing an age-related disease based on the subject's biological age; and optionally select a suitable dietary, pharmacological, or lifestyle regime for the subject based on the subject's risk of developing an age-related disease based on the subject's biological age, as determined by the present method. The computer program product may also be given additional parameters or characteristics for the subject. Suitably, the computer program product comprises a computer-readable medium comprising said computer-implementable instructions.

In one aspect, the present invention provides a computer system for determining the biological age of an animal as described herein. Suitably, the computer program product comprises a computer-readable medium comprising said computer-implementable instructions.

In another aspect, the present invention provides a computer system for determining the biological age of an animal subject; and optionally select a suitable dietary, pharmacological, or lifestyle regime for the subject based on the subject's biological age, as determined by the present method.

In another aspect, the present invention provides a computer system for determining whether an animal subject is at a risk of developing an age-related disease based on the subject's biological age; and optionally select a suitable dietary, pharmacological, or lifestyle regime for the subject based on the subject's risk of developing an age-related disease based on the subject's biological age, as determined by the present method.

In one embodiment, the user inputs into the computer system levels in an animal subject of five or biomarkers as defined herein. The computer system then processes this information and provides a determination of biological age. Alternatively, the computer system then processes this information and provides a determination of a suitable dietary, pharmacological, or lifestyle regime for the dog based on the biological age, as determined by the present method.

The computer system or device may generally be a server on a network. However, any computer system or device may be used as long as it can process biomarker data and/or additional parameters or characteristic data using a processor, a central processing unit (CPU) or the like. The computer system or device may, for example, be a smartphone, a tablet terminal or a personal computer and output information indicating the determined biological age of the subject or a determination of a suitable lifestyle or dietary regime for the subject based on the determined biological age.

Various preferred features and embodiments of the present invention will now be described by way of non-limiting examples. This disclosure is not limited by the exemplary methods and materials disclosed herein, and any methods and materials similar or equivalent to those described herein can be used in the practice or testing of embodiments of this disclosure. The skilled person will understand that they can combine all features of the invention disclosed herein without departing from the scope of the invention as disclosed.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes”, “containing”, or “contains”, and are inclusive or open-ended and do not exclude additional, non-recited members, elements or steps. The terms “comprising”, “comprises” and “comprised of” also include the term “consisting of”.

Numeric ranges are inclusive of the numbers defining the range. As used herein the term “about” means approximately, in the region of, roughly, or around. When the term “about” is used in conjunction with a numerical value or range, it modifies that value or range by extending the boundaries above and below the numerical value(s) set forth. In general, the terms “about” and “approximately” are used herein to modify a numerical value(s) above and below the stated value(s) by 10%.

The publications discussed herein are provided solely for their disclosure prior to the filing date of the present application. Nothing herein is to be construed as an admission that such publications constitute prior art to the claims appended hereto.

All publications mentioned in the specification are herein incorporated by reference.

### EXAMPLES

The invention will now be further described by way of examples, which are meant to serve to assist one of ordinary skill in the art in carrying out the invention and are not intended in any way to limit the scope of the invention.

### Illustrative Method for Determining Biological Age in Dogs Using Proteomics

### Dataset Description

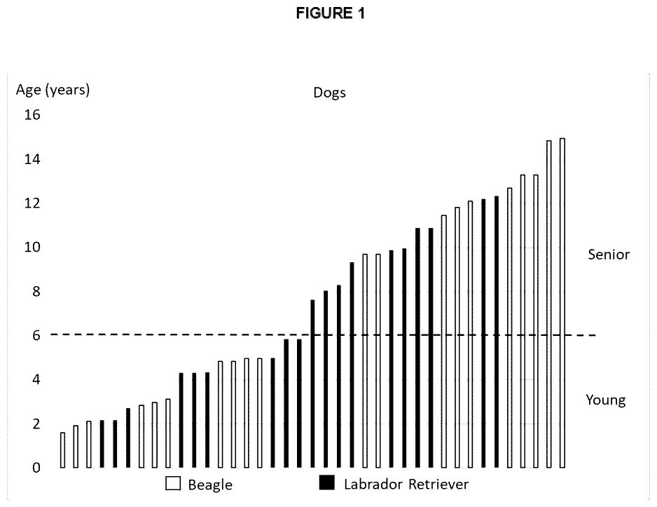

A biological age determination tool using proteomic data was developed using a pet cohort composed of 39 dogs. The cohort was split into two groups. The young group was based on 2-6 year-olds. The senior group was based on 7+ year olds (FIG. 1).

Dogs were fed a complete and balanced diet at 100% of their estimated MERs for 5 weeks to stabilize their body weight (less than 5% changes in three weeks) by adjusting their food intake and standardize their metabolomic profiles. At the end of 5 weeks a fasted blood sample was collected. Serum samples were aliquoted into two tubes with 250 ul per tube and two tubes with 125 ul per tube.

39 dog serum samples (plus 3 dog quality control samples—a mixture of multiple dog samples) were used in the study.

### Proteomic Analysis

Proteomic profiling was performed on the SomaScan discovery platform (SOMALogic, Boulder, CO; Davies D R et al., 2012. Proc Natl Acad Sci USA. 109 (49): 19971-6). The protein-capture reagents were aptamers—short single-stranded DNA sequences—and through equilibration binding as well as removal of unbound aptamers and unbound proteins, the concentration of proteins in the matrix were transformed into a relative quantity of aptamers. The aptamer quantity was then measured by hybridization to microarrays.

The V4 panel of SOMAscan assay, targeted to 4.9 k human proteins, was used with canine serum samples alongside species-specific Quality Control samples (pooled sera from healthy dogs). The proteomics data were expressed as abundance in relative fluorescence units (RFU).

Sample data were first normalized to remove hybridization variation within a run, followed by median normalization across calibrator samples to remove other potential assay biases within the run. Overall scaling was then performed on a per-plate basis to remove overall intensity differences between runs. Samples were then normalized against an intra-plate, study-specific plate reference. Finally, analytes were screened using the dog QCs on Signal-to-Noise ratio (above 2), F-stats (p<0.05) and intra-plate CV (less than 10%). A total of 2,408 proteins passed screening for the canine study.

### Age Modelling Using Proteomics

### Data Preprocessing

The Python library Pandas (Mckinney W., 2010. Proc. of the 9^{th} Python in Science Conf. 56-61) was used for data cleaning, filtering, and transforming omics datasets to make them suitable for machine learning (ML) models. The relative abundance of proteins from the 39 dog samples was provided for age prediction and feature importance assessment that identifies key biomarkers using ML regression ensemble models such as random forest and AdaBoost. The dog proteomics consist of 2,408 protein features. After initial cleaning of the raw data, the dataset was transformed and scaled using min-max normalization method from the scikit learn (Fabian P et al., 2011. Journal of Machine Learning Research. 2825-2830) preprocessing package, Z-score, and log 2 normalization methods.

$$Min Max Scaler = \frac{value - min}{max - min} \qquad ( 1 )$$

$$Z - score = \frac{value - μ}{σ} \qquad ( 2 )$$

where μ is the mean value of the feature and σ is the standard deviation of the feature. The dataset was then split into training and testing sets using the stratified random sampling method, which ensures that the class distribution is preserved in both sets. The 80-20 rule was used, which means that 80% of the data is allocated for training and 20% for testing.

### Regression Machine Learning Method: Age, Continuous Prediction

Scikit-learn 1.3.2 (Fabian, 2011) was used for ML workflow to predict the chronological age of dogs from the proteomics dataset created from the early integration method. In this study, two ensemble learning methods, the Random Forest (RF) regressor and the AdaBoost (AB) regressor, were used for the regression task.

The regression model performance is highly affected by hyperparameters. The grid search cross-validation was used which is the algorithm for finding the optimal combination of the hyperparameters for the regression model. The grid search cross-validation involves defining a grid of possible values for each hyperparameter and evaluating the model performance on a validation set for each combination of hyperparameters. An example random forest parameter grid is:

```
param_grid = { [symbol]
  bootstrap [symbol] : [True], [symbol]
  max_depth [symbol] : [ [symbol] None [symbol] , 1, 2, 3, 4, 5], [symbol]
  max_features [symbol] : [ [symbol] auto [symbol] , 1, 2, 3, 4, 5], [symbol]
  min_samples_leaf [symbol] : [1, 2, 3, 4, 5], [symbol]
  min_samples_split [symbol] : [2, 5, 10], [symbol]
  n_estimators [symbol] : [10, 20, 100, 200]
} [symbol]
indicates data missing or illegible when filed
```

The dataset was stratified according to two age groups, young and senior, to ensure both training and testing sets have an equal ratio of young and senior individuals. The random state number was set to 42 and the train_test_split function was utilized in the following manner.

$$X\_train , X\_test , y\_train , y\_test = train\_test \_split \text{} ( X , y , test\_size = 0.2 , stratify = age\_groups , random\_state = 42 ) \qquad ( 3 )$$

AdaBoostRegressor Algorithm: Grid search cross-validation was utilized to identify the optimal parameters for AdaBoost model. In the model, the number of estimators was set to 200, max_depth parameters defaults to 3, and random_state number was set to 0.

$$regr = AdaBoostRegressor ( n\_estimators = 200 , \text{} learning\_rate = 1. , loss = ‘ linear ’ , random\_state = 0 ) \qquad ( 4 )$$

To ensure consistent results (i.e., r² value 0.84) when using the dataset, all specified random_state values should be set as shown in (3) and (4). The importance of features can be determined using feature_importances_attribute. The top 24 proteins were identified from a pool of 50000 and the model reconstructed using the same algorithm to achieve the final r² value of 0.84.

### Input Data Sequencing

A selection of proteins was selected for input into the model and assembled into the input file, and their relative abundance values were inputted as a NumPy array into the model (2).

Classification Machine Learning Method: Young Vs. Senior, Categorical Prediction

The supervised ML classification was performed for classifying groups between young (under 7 years old) and senior dogs (above 7 years old) from the proteomics dataset. The partial least square (PLS2—two dependent variables: young vs. senior) algorithm was used mainly for the dimensionality reduction and the feature extraction. Then, the support vector machine (SVM) was used as a classifier to separate the two groups (senior and young). The SVM model performance was evaluated using the accuracy and F1-score metrics from the confusion matrix on the test set. The middle-integration approach was performed using the DIABLO method in the mixOmics package (Le Cao, 2009) from R 4.2.2. The DIABLO method constructs latent components like PLS2 but the sum of covariance between all pairs of omics data sets is maximized.

### Results

### Continuous Age Model

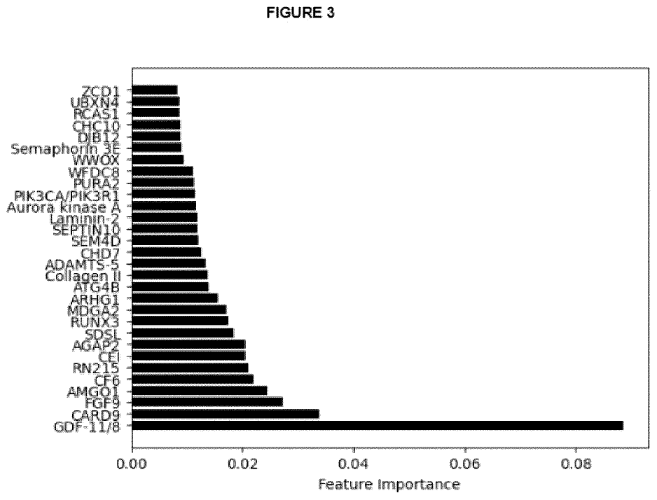

With the proteomics dataset, the AdaBoost regressor showed the best performance in terms of the r² value (0.68). A set of optimal hyperparameter values was obtained using grid search with 5-fold cross-validation list. The top 24 proteins, ranked in order of importance, for the canine continuous age model are shown in FIG. 3 and Table 4.

TABLE 4

| | | | UniProt | UniProt |
|---|---|---|---|---|
| Rank | Percent | Top Proteins | (Human) | (Canine) |
| 1 | 0.089 | GDF-11/8 | O95390 | A0A8I3MVU8 |
| | | | O14793 | Q6UKZ8 |
| 2 | 0.034 | CARD9 | Q9H257 | A0A8I3N256 |
| 3 | 0.027 | FGF9 | P31371 | A0A8C0TNC4 |
| 4 | 0.024 | AMGO1 | Q86WK6 | A0A8I3MLU9 |
| 5 | 0.022 | CF6 | P18859 | A0A0B2VY56 |
| 6 | 0.021 | RN215 | Q9Y6U7 | A0A8P0NEJ2 |
| 7 | 0.021 | CEI | Q86SI9 | A0A8C0N5G3 |
| 8 | 0.021 | AGAP2 | Q99490 | A0A8I3NL11 |
| 9 | 0.018 | SDSL | Q96GA7 | A0A8C0PJ89 |
| 10 | 0.017 | RUNX3 | Q13761 | A0A8C0PLB3 |
| 11 | 0.017 | MDGA2 | Q7Z553 | A0A8P0PIE7 |
| 12 | 0.016 | ARHG1 | Q92888 | A0A8C0QQ66 |
| 13 | 0.014 | ATG4B | Q9Y4P1 | A0A8C0PRI8 |
| 14 | 0.014 | Collagen II | P02458 | A0A8C0PFU8 |
| 15 | 0.013 | ADAMTS-5 | Q9UNA0 | A0A8P0SDL0 |
| 16 | 0.013 | CHD7 | Q9P2D1 | A0A8C0SX81 |
| 17 | 0.012 | SEM4D | Q92854 | A0A8C0PK44 |
| 18 | 0.012 | SEPTIN10 | Q9P0V9 | A0A8C0RTA3 |
| 19 | 0.012 | Laminin-2 | P24043 | A0A8I3RRA4A0A8I3PL58 |
| | | | P07942 | A0A8C0M627 |
| | | | P11047 | |
| 20 | 0.012 | Aurora | O14965 | A0A8I3S724 |
| | | kinase A | | |
| 21 | 0.012 | PIK3CA/ | P42336 | A0A5F4C2B1 |
| | | PIK3R1 | P27986 | A0A8I3N971 |
| 22 | 0.011 | PURA2 | P30520 | A0A8C0P3B8 |
| 23 | 0.011 | WFDC8 | Q8IUA0 | A0A8I3NZM8 |
| 24 | 0.010 | WWOX | Q9NGC7 | A0A8I3P991 |

Table 4. The AdaBoostRegressor model identifies the significance of each feature. Features with an importance score above the threshold of 0.01 are considered significant, such as the WWOX feature in this case.

Continuous Age Model with Top 24 Features Exhibits High Performance

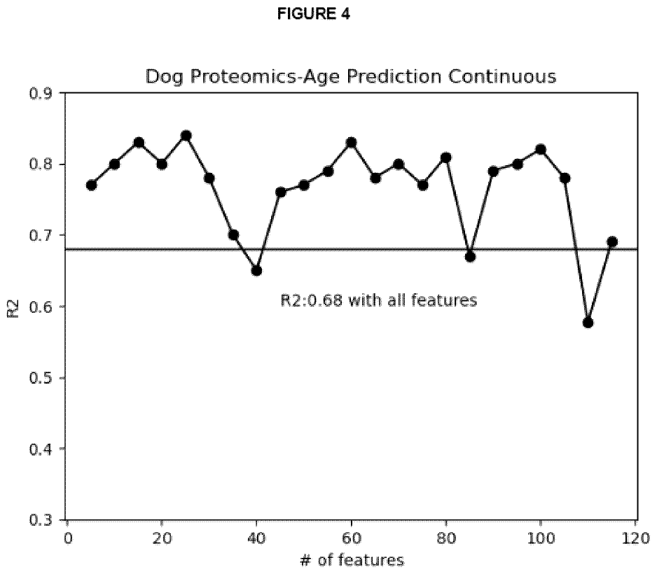

After reconstructing the model with the top 24 features, the model performance increased with r² value of 0.84. The r-squared value was plotted as a function of the number of features as shown in FIG. 4.

New dog samples from another study were used to test the AdaBoost regression model. Only the 24 features of each sample were extracted as listed in Table 2. The predicted proteo-age of these subjects compared to their chronological age is summarized in Table 5.

TABLE 5

| Samples | Chronological Age | Proteo-Age (predicted) | Delta age |
|---|---|---|---|
| Dog 1 | 11.15 | 11.30 | +0.15 |
| Dog 2 | 9.48 | 7.50 | −1.98 |
| Dog 3 | 8.24 | 9.51 | +1.3 |

Table 5. The AdaBoost regression model predicted the proteo-age of new unseen samples from another study. Delta age is the difference between chronological age and proteo-age.

Continuous Age Models with Randomly Selected Features from the Top 10 Features Exhibit High Performance

The models were reconstructed using 5 randomly selected features from the top 10 features. The resulting r² values are displayed in Table 6.

TABLE 6

| Five Random Features | r² value |
|---|---|
| CEI, FGF9, AGAP2, SDSL, DGF-11/8 | 0.78 |
| AMGO1, CARD9, SDSL, RUNX3, FGF9 | 0.79 |
| FGF9, AGAP2, RUNX3, SDSL, CF6 | 0.86 |
| CARD9, RN215, AMGO1, SDSL, CEI | 0.77 |
| RUNX3, RN215, SDSL, FGF9, CEI | 0.79 |
| RN215, AMGO1, CF6, CEI, SDSL | 0.65 |
| SDSL, RN215, AGAP2, CF6, CEI | 0.76 |

Table 6. The AdaBoost Regressor model with randomly selected top 5 features results in high r² values.

### Dietary Treatment

Two different dietary interventions were provided to two dogs whose proteo-age was predicted by the AdaBoost regression model. The proteo-age was predicted again following the dietary interventions. The proteo-age of both dogs before and after providing the diets is summarized in Table 7.

TABLE 7

| | Proteo-age at | Proteo-Age after 6 months |
|---|---|---|
| Example | Base Line (years) | of feeding (years) |
| Dog 1/Diet A | 10.89 | 7.49 |
| Dog2/Diet B | 7.51 | 7.98 |

Table 7. Two different dietary treatments affect proteo-age after 6 months, which was predicted by AdaBoost regression model.

### Ridge Regression Model Comparison

The AdaBoost model's performance was compared with a linear regression model-ridge regression. The proteo-age was predicted based on the top 24 important features which were obtained from the AdaBoost model. The ridge regression estimator was obtained by adding a penalty term to the ordinary least square (OLS) estimator. It aims to reduce the variance of coefficient estimates in multiple linear regression models and provides a way to handle multicollinearity by introducing a penalty term that shrinks the coefficient toward zero. The mathematical formula for the linear least squares with the regularization is as follows:

$${‖ y - {X}_{w} ‖}_{2}^{2} + alpha * {‖ w ‖}_{2}^{2} \qquad ( 5 )$$

where y represents the response variable, X_{w} is the feature matrix, alpha is the regularization parameter controlling the strength of the penalty, and w is the vector of regression coefficients.

Scikit-learn machine learning library was used to build the ridge regressor for this study,

$$ridge\_model = linear\_model . RidgeCV ( alpha = np . logspace ( - 6 , 6 , 13 ) ) \qquad ( 6 )$$

RidgeCV implements ridge regression with Leave-One-Out cross-validation of the alpha parameter.

Feature weight vectors were calculated by using the coef_attribute as follows:

$$ridge\_model . coef\_ \qquad ( 7 )$$

The linear regression—ridge regression—was performed using various numbers of features. Increasing numbers of features were used in the regression model in order of their importance as identified by the AdaBoost model. The r² value was shown to vary depending on the number of features included in the regression model.

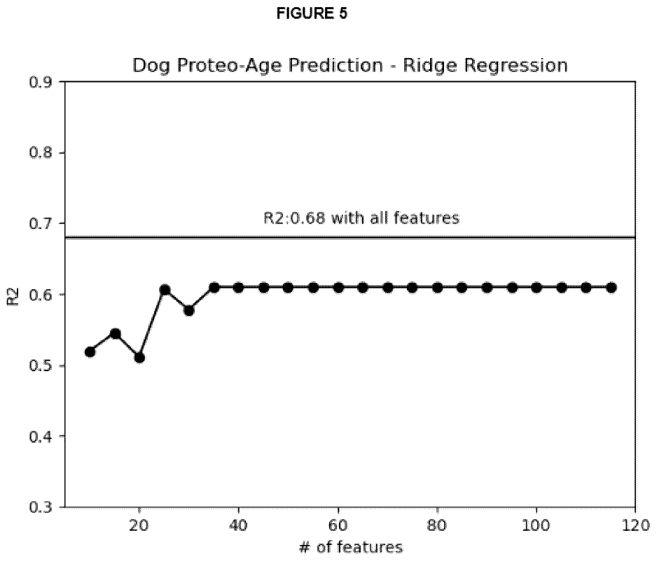

As is shown in FIG. 5, the r² value of the ridge regression model reaches a value of 0.61 when using the top 24 most important features as identified using the AdaBoost model. This is compared to the r² value of 0.68 reached by an AdaBoost model using all features.

The feature weight vectors as determined by ridge regression for the top 24 features identified by the AdaBoost are provided in Table 8 below.

TABLE 8

| Feature | Weight Vector (w) |
|---|---|
| GDF-11/8 | −70.696704 |
| CARD9 | −35.443741 |
| FGF9 | −12.791139 |
| AMGO1 | −6.443659 |
| CF6 | 19.785827 |
| RN215 | 127.688649 |
| CEI | 64.957304 |
| AGAP2 | −2.691673 |
| SDSL | 3.120605 |
| RUNX3 | −9.192050 |
| MDGA2 | −28.412413 |
| ARHG1 | −48.102437 |
| ATG4B | 9.725143 |
| Collagen II | −10.359702 |
| ADAMTS-5 | 52.044874 |
| CHD7 | 12.568455 |
| SEM4D | 6.320015 |
| SEPTIN10 | −27.857657 |
| Laminin-2 | 6.272020 |
| Aurora-Kinase A | −18.468224 |
| PIK3CA/PIK3E1 | −23.510377 |
| PURA2 | 8.573350 |
| WFDC8 | −17.899146 |
| WWOX | 0.812681 |

Table 8: Feature weight vectors as determined by ridge regression for top 24 AdaBoost features.

A linear regression model was capable of achieving high r² values when using groups of features identified to have high importance by the AdaBoost ML model.

Categorical Young vs. Senior Age Model

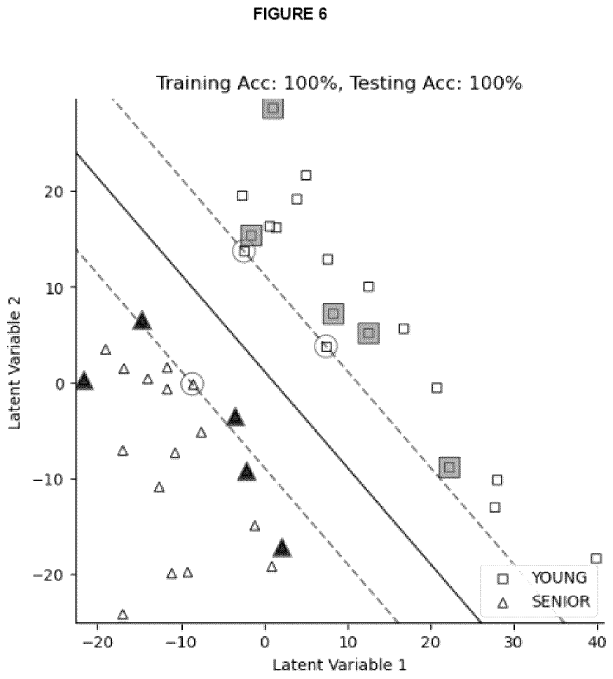

SVM classification was carried out on the proteins to develop a model representing young vs senior canines was developed (FIG. 6). The top proteins, in order of importance, for the canine young versus senior age classification model are shown in Table 3.

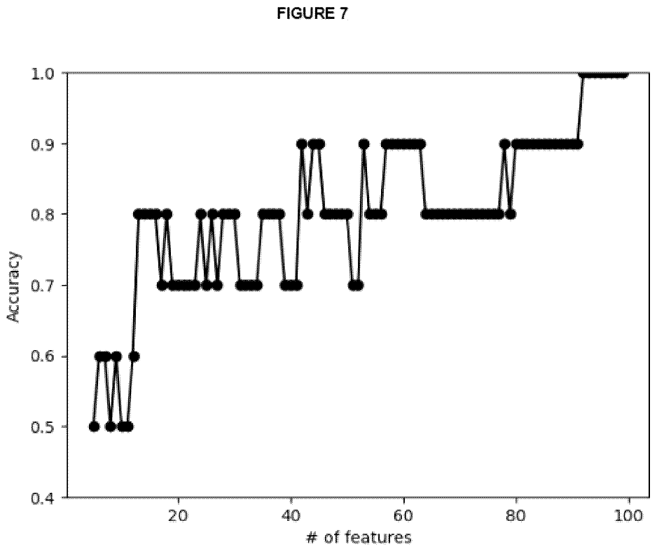

The accuracy of the canine young versus senior classification model was measured for models with varying numbers of the most important features included. The accuracy of the classification model as a function of the number of features is shown in FIG. 7, and Table 9.

TABLE 9

| | | | | ML Accuracy |
|---|---|---|---|---|
| Rank by | | UniProt | UniProt | by # of |
| P-value | Top Proteins | (Human) | (Canine) | features |
| 1 | NTRI | Q9P121 | A0A8I3MR44 | NA |
| 2 | ADAMTS-5 | Q9UNA0 | A0A8P0SDL0 | NA |
| 3 | ARHG1 | Q92888 | A0A8C0QQ66 | NA |
| 4 | RUNX3 | Q13761 | A0A8C0PLB3 | NA |
| 5 | CEI | Q86SI9 | A0A8C0N5G3 | 0.5 |
| 6 | AGAP2 | Q99490 | A0A8I3NL11 | 0.6 |
| 7 | NXF1 | Q9UBU9 | A0A8I3N3V9 | 0.6 |
| 8 | IGDC4 | Q8TDY8 | A0A8C0K7I6 | 0.5 |
| 9 | Laminin-2 | P24043 | A0A8I3RRA4A0A8I3PL58 | 0.6 |
| | | P07942 | A0A8C0M627 | |
| | | P11047 | | |
| 10 | NTRI | Q9P121 | A0A8I3MR44 | 0.5 |
| 11 | RCD1 | Q92600 | A0A0B2UZ33 | 0.5 |
| 12 | CARD9 | Q9H257 | A0A8I3N256 | 0.6 |
| 13 | SRCN1 | P12931 | A0A8I3SBF9 | 0.8 |
| 14 | NMES1 | Q9C002 | A0A8C0N9H4 | 0.8 |
| 15 | sE-Selectin | P16581 | A0A8I3MRV9 | 0.8 |
| 16 | AGO1 | Q9UL18 | A0A8C0RZU0 | 0.8 |
| 17 | Calcineurin | Q08209 | A0A8I3PS73 | 0.7 |
| | | P63098 | A0A8I3PQY6 | |
| 18 | GDF-11/8 | O95390 | A0A8I3MVU8 | 0.8 |
| | | O14793 | Q6UKZ8 | |
| 19 | STK24 | Q9Y6E0 | A0A8I3S1Q3 | 0.7 |
| 20 | Ezrin | P15311 | A0A8C0NAG2 | 0.7 |
| 21 | Thioredoxin reductase 1 | Q16881 | A0A8C0K7A3 | 0.7 |
| 22 | HSP 70 | P0DMV8 | A0A0B2V5X6 | 0.7 |
| 23 | BOC | Q9BWV1 | A0A8C0TJR0 | 0.7 |
| 24 | CNOT1 | A5YKK6 | A0A8I3MQ47 | 0.8 |
| 25 | MARCKSL1 | P49006 | A0A8C0TGC8 | 0.7 |
| 26 | MK10 | P53779 | A0A8C0Z6V9 | 0.8 |
| 27 | PABP4 | Q13310 | A0A8I3N997 | 0.7 |
| 28 | CSNK1G2 | P78368 | A0A8C0L0H2 | 0.8 |
| 29 | Moesin | P26038 | A0A8C0TC91 | 0.8 |
| 30 | PGCB | Q96GW7 | H6VX53 | 0.8 |
| 31 | TIM50 | Q3ZCQ8 | A0A8I3MU85 | 0.7 |
| 32 | MFAP5 | Q13361 | A0A8C0PCR8 | 0.7 |
| 33 | SYTL4 | Q96C24 | A0A8C0NF93 | 0.7 |
| 34 | SEPTIN10 | Q9P0V9 | A0A8C0RTA3 | 0.7 |
| 35 | PKB gamma | Q9Y243 | A0A8C0NWA6 | 0.8 |
| 36 | PCDH8 | O95206 | A0A8C0L1A7 | 0.8 |
| 37 | CAMK2B | Q13554 | A0A8C0RD52 | 0.8 |
| 38 | MEK1 | Q02750 | A0A8C0NAT7 | 0.8 |
| 39 | HSP 10 | P61604 | A0A8C0KIA5 | 0.7 |
| 40 | OAF | Q86UD1 | A0A8C0M6K0 | 0.7 |
| 41 | MDGA2 | Q7Z553 | A0A8P0PIE7 | 0.7 |
| 42 | BARK1 | P25098 | A0A8I3NE91 | 0.9 |
| 43 | JNK2 | P45984 | A0A8C0QAU7 | 0.8 |
| 44 | LYN | P07948 | A0A8I3PUN0 | 0.9 |
| 45 | NUCB1 | Q02818 | A0A8C0PVG8 | 0.9 |
| 46 | MAPK14 | Q16539 | A0A8C0SYM9 | 0.8 |
| 47 | STAT3 | P40763 | A0A8C0QJT6 | 0.8 |
| 48 | HMHA1 | Q92619 | A0A8C0QDY6 | 0.8 |
| 49 | FGF9 | P31371 | A0A8C0TNC4 | 0.8 |
| 50 | RPAB4 | P53803 | A0A8C0NDJ7 | 0.8 |
| 51 | CAMK2A | Q9UQM7 | A0A8I3NIS8 | 0.7 |
| 52 | LCN8 | Q6JVE9 | A0A8I3MWP0 | 0.7 |
| 53 | RIR2B | Q7LG56 | A0A8C0NXM7 | 0.9 |
| 54 | MP2K4 | P45985 | A0A8I3MIB2 | 0.8 |
| 55 | RHG25 | P42331 | A0A8C0P719 | 0.8 |
| 56 | PP1G | P36873 | Q8MJ46 | 0.8 |
| 57 | CHKB | Q9Y259 | A0A8I3PU66 | 0.9 |
| 58 | MYCBP | Q99417 | A0A8C0M044 | 0.9 |
| 59 | PKB | P31749 | A0A8C0MEP1 | 0.9 |
| 60 | WFDC8 | Q8IUA0 | A0A8I3NZM8 | 0.9 |
| 61 | WWP1 | Q9H0M0 | A0A8C0MK13 | 0.9 |
| 62 | UBS3B | Q8TF42 | A0A8C0LWY3 | 0.9 |
| 63 | CAMK2D | Q13557 | A0A8C0NZM8 | 0.9 |
| 64 | DUSP13 | Q6B8I1 | A0A8C0NVL5 | 0.8 |
| 65 | HSP 90a | P07900 | A0A8I3MR78 | 0.8 |
| 66 | GSK-3 alpha | P49840 | A0A8I3MIZ4 | 0.8 |
| 67 | CSNK1G2 | P78368 | A0A8C0L0H2 | 0.8 |
| 68 | RN215 | Q9Y6U7 | A0A8P0NEJ2 | 0.8 |
| 69 | COMMD7 | Q86VX2 | A0A8C0Q728 | 0.8 |
| 70 | M2-PK | P14618 | A0A8C0YZD9 | 0.8 |
| 71 | AP4M1 | O00189 | A0A8I3RQU5 | 0.8 |
| 72 | MK08 | P45983 | A0A8I3NY64 | 0.8 |
| 73 | HSP70 protein 8 | P11142 | A0A8C0KE17 | 0.8 |
| 74 | Histidyl-tRNA synthetase | P12081 | A0A8I3MNZ5 | 0.8 |
| 75 | TrkB | Q16620 | Q6JDI7 | 0.8 |
| 76 | PIK3CA/PIK3R1 | P42336 | A0A5F4C2B1 | 0.8 |
| | | P27986 | A0A8I3N971 | |
| 77 | MESD2 | Q14696 | A0A0B2VNA1 | 0.8 |
| 78 | PGAM2 | P15259 | A0A8C0TSU2 | 0.9 |
| 79 | KBP | Q96EK5 | A0A8I3MUW8 | 0.8 |
| 80 | CSK | P41240 | A0A8C0RH82 | 0.9 |
| 81 | ETS2 | P15036 | A0A8C0RTI9 | 0.9 |
| 82 | OST48 | P39656 | Q05052 | 0.9 |
| 83 | SUMF2 | Q8NBJ7 | A0A8I3NQ68 | 0.9 |
| 84 | USO1 | O60763 | A0A8I3Q5G1 | 0.9 |
| 85 | Angiostatin | P00747 | P80009 | 0.9 |
| 86 | FER | P16591 | Q9TTY2 | 0.9 |
| 87 | NAA10 | P41227 | A0A0B2W6H8 | 0.9 |
| 88 | PDIA5 | Q14554 | A0A8C0TL18 | 0.9 |
| 89 | NMI | Q13287 | A0A8P0NZK0 | 0.9 |
| 90 | SEM4D | Q92854 | A0A8C0PK44 | 0.9 |
| 91 | NCC27 | O00299 | A0A8C0L4R5 | 0.9 |
| 92 | COL11A2 | P13942 | A0A8I3PB22 | 1 |
| 93 | KSYK | P43405 | A0A8C0M271 | 1 |
| 94 | ARHGP | Q86VW2 | A0A8C0RE44 | 1 |

Table 9: Top 94 proteins—Canine Categorical Young vs. Senior Model

The accuracy of the model is 0.6 when using only 6 of the most important features. The accuracy of the model is 0.8 when using only 13 of the most important features. The accuracy of the model is 0.9 when using 42 of the most important features. The model accuracy reaches 1 when using 92 of the most important features.

The accuracy value of the SVM model was measured when using 5 randomly selected features from the top 10 most important features. The accuracy of the models using these features is shown in Table 10.

TABLE 10

| Five Random Features | Accuracy (%) |
|---|---|
| Laminin-2, NXF1, CEI, NTRI, ARHG1 | 66 |
| NTRI, RUNX3, NXF1, AGAP2, ARHG1 | 76 |
| ARHG1, AGAP2, CEI, NXF1, RUNX3 | 72 |
| ADAMTS-5, NTRI, IGDC4, CEI, NXF1 | 76 |
| CEI, RUNX3, NTRI, AGAP2, ADAMTS-5 | 79 |
| NXF1, NTRI, IGDC4, RUNX3, CEI | 69 |
| RUNX3, NTRI, IGDC4, Laminin-2, NXF1 | 72 |
| Laminin-2, CEI, ARHG1, IGDC4, NTRI | 66 |

Table 10: SVM model accuracy when using 5 randomly selected features from the top 10 most important features.

The accuracy of the classification model was tested on 29 new dog samples obtained from another study. Biomarker levels of the 24 features identified to be of most importance were determined from each new sample and supplied to the model. The categorical age predicted by the model for each new dog, and the dog's age category based on chronological age are shown in Table 11. The overall accuracy of the classification model using 24 features on an unseen sample set was 0.72.

TABLE 11

| Samples | Actual | Predicted | Classification |
|---|---|---|---|
| Dog 1 | Young | Young | Correct |
| Dog 2 | Young | Senior | Incorrect |
| Dog 3 | Senior | Young | Incorrect |
| Dog 4 | Young | Young | Correct |
| Dog 5 | Senior | Senior | Correct |
| Dog 6 | Young | Young | Correct |
| Dog 7 | Senior | Senior | Correct |
| Dog 8 | Senior | Senior | Correct |
| Dog 9 | Senior | Young | Incorrect |
| Dog 10 | Young | Senior | Incorrect |
| Dog 11 | Young | Young | Correct |
| Dog 12 | Young | Young | Correct |
| Dog 13 | Young | Young | Correct |
| Dog 14 | Senior | Young | Incorrect |
| Dog 15 | Senior | Senior | Correct |
| Dog 16 | Young | Young | Correct |
| Dog 17 | Young | Young | Correct |
| Dog 18 | Young | Young | Correct |
| Dog 19 | Young | Senior | Incorrect |
| Dog 20 | Young | Young | Correct |
| Dog 21 | Senior | Young | Incorrect |
| Dog 22 | Young | Young | Correct |
| Dog 23 | Young | Young | Correct |
| Dog 24 | Young | Young | Correct |
| Dog 25 | Senior | Young | Incorrect |
| Dog 26 | Young | Young | Correct |
| Dog 27 | Young | Young | Correct |
| Dog 28 | Young | Young | Correct |
| Dog 29 | Young | Young | Correct |

Table 11: Accuracy of the classification model predicting age category of dogs in an unseen sample set.

TABLE 1

| | Uniprot ID | Uniprot ID |
|---|---|---|
| Protein | (Human) | (Canine) |
| CARD9 | Q9H257 | A0A8I3N256 |
| GDF-11/8 | O95390 | A0A8I3MVU8 |
| | O14793 | Q6UKZ8 |
| FGF9 | P31371 | A0A8C0TNC4 |
| AMGO1 | Q86WK6 | A0A8I3MLU9 |
| CF6 | P18859 | A0A0B2VY56 |
| RN215 | Q9Y6U7 | A0A8P0NEJ2 |
| CEI | Q86SI9 | A0A8C0N5G3 |
| AGAP2 | Q99490 | A0A8I3NL11 |
| SDSL | Q96GA7 | A0A8C0PJ89 |
| RUNX3 | Q13761 | A0A8C0PLB3 |
| MDGA2 | Q7Z553 | A0A8P0PIE7 |
| ARHG1 | Q92888 | A0A8C0QQ66 |
| ATG4B | Q9Y4P1 | A0A8C0PRI8 |
| Collagen II | P02458 | A0A8C0PFU8 |
| ADAMTS-5 | Q9UNA0 | A0A8P0SDL0 |
| CHD7 | Q9P2D1 | A0A8C0SX81 |
| SEM4D | Q92854 | A0A8C0PK44 |
| SEPTIN10 | Q9P0V9 | A0A8C0RTA3 |
| Laminin-2 | P24043 | A0A8I3RRA4A0A8I3PL58 |
| | P07942 | A0A8C0M627 |
| | P11047 | |
| Aurora kinase A | O14965 | A0A8I3S724 |
| PIK3CA/PIK3R1 | P42336 | A0A5F4C2B1 |
| | P27986 | A0A8I3N971 |
| PURA2 | P30520 | A0A8C0P3B8 |
| WFDC8 | Q8IUA0 | A0A8I3NZM8 |
| WWOX | Q9NGC7 | A0A8I3P991 |
| Semaphorin 3E | Q14563 | A0A8I3QD90 |
| DJB12 | Q9NXW2 | A0A0B2VFS4 |
| CHC10 | Q8WYQ3 | A0A8C0TAW0 |
| RCAS1 | O00559 | A0A8I3NSV4 |
| UBXN4 | Q92575 | A0A8C0SKX6 |
| ZCD1 | Q9NZ45 | A0A0B2V3B6 |
| NTRI | Q9UNA0 | A0A8I3MR44 |
| NXF1 | Q9UBU9 | A0A8I3N3V9 |
| IGDC4 | Q8TDY8 | A0A8C0K716 |
| RCD1 | Q92600 | A0A0B2UZ33 |
| SRCN1 | P12931 | A0A8I3SBF9 |
| NMES1 | Q9C002 | A0A8C0N9H4 |
| sE-Selectin | P16581 | A0A8I3MRV9 |
| AGO1 | Q9UL18 | A0A8C0RZU0 |
| Calcineurin | Q08209 | A0A8I3PS73 |
| | P63098 | A0A8I3PQY6 |
| STK24 | Q9Y6E0 | A0A8I3S1Q3 |
| Ezrin | P15311 | A0A8C0NAG2 |
| Thioredoxin | Q16881 | A0A8C0K7A3 |
| reductase 1 | | |
| HSP 70 | P0DMV8 | A0A0B2V5X6 |
| BOC | Q9BWV1 | A0A8C0TJR0 |
| CNOT1 | A5YKK6 | A0A8I3MQ47 |
| MARCKSL1 | P49006 | A0A8C0TGC8 |
| MK10 | P53779 | A0A8C0Z6V9 |
| PABP4 | Q13310 | A0A8I3N997 |
| CSNK1G2 | P78368 | A0A8C0L0H2 |
| Moesin | P26038 | A0A8C0TC91 |
| PGCB | Q96GW7 | H6VX53 |
| TIM50 | Q3ZCQ8 | A0A8I3MU85 |
| MFAP5 | Q13361 | A0A8C0PCR8 |
| SYTL4 | Q96C24 | A0A8C0NF93 |
| PKB gamma | Q9Y243 | A0A8C0NWA6 |
| PCDH8 | O95206 | A0A8C0L1A7 |
| CAMK2B | Q13554 | A0A8C0RD52 |
| MEK1 | Q02750 | A0A8C0NAT7 |
| HSP 10 | P61604 | A0A8C0KIA5 |
| OAF | Q86UD1 | A0A8C0M6K0 |
| BARK1 | P25098 | A0A8I3NE91 |
| JNK2 | P45984 | A0A8C0QAU7 |
| LYN | P07948 | A0A8I3PUN0 |
| NUCB1 | Q02818 | A0A8C0PVG8 |
| MAPK14 | Q16539 | A0A8C0SYM9 |
| STAT3 | P40763 | A0A8C0QJT6 |
| HMHA1 | Q92619 | A0A8C0QDY6 |
| RPAB4 | P53803 | A0A8C0NDJ7 |
| CAMK2A | Q9UQM7 | A0A8I3NIS8 |
| LCN8 | Q6JVE9 | A0A8I3MWP0 |
| RIR2B | Q7LG56 | A0A8C0NXM7 |
| MP2K4 | P45985 | A0A8I3MIB2 |
| RHG25 | P42331 | A0A8C0P719 |
| PP1G | P36873 | Q8MJ46 |
| CHKB | Q9Y259 | A0A8I3PU66 |
| MYCBP | Q99417 | A0A8C0M044 |
| PKB | P31749 | A0A8C0MEP1 |
| WWP1 | Q9H0M0 | A0A8C0MK13 |
| UBS3B | Q8TF42 | A0A8C0LWY3 |
| CAMK2D | Q13557 | A0A8C0NZM8 |
| DUSP13 | Q6B8I1 | A0A8C0NVL5 |
| HSP 90a | P07900 | A0A8I3MR78 |
| GSK-3 alpha | P49840 | A0A8I3MIZ4 |
| COMMD7 | Q86VX2 | A0A8C0Q728 |
| M2-PK | P14618 | A0A8C0YZD9 |
| AP4M1 | O00189 | A0A8I3RQU5 |
| MK08 | P45983 | A0A8I3NY64 |
| HSP70 protein 8 | P11142 | A0A8C0KE17 |
| Histidyl-tRNA | P12081 | A0A8I3MNZ5 |
| synthetase | | |
| TrkB | Q16620 | Q6JDI7 |
| MESD2 | Q14696 | A0A0B2VNA1 |
| PGAM2 | P15259 | A0A8C0TSU2 |
| KBP | Q96EK5 | A0A8I3MUW8 |
| CSK | P41240 | A0A8C0RH82 |
| ETS2 | P15036 | A0A8C0RTI9 |
| OST48 | P39656 | Q05052 |
| SUMF2 | Q8NBJ7 | A0A8I3NQ68 |
| USO1 | O60763 | A0A8I3Q5G1 |
| Angiostatin | P00747 | P80009 |
| FER | P16591 | Q9TTY2 |
| NAA10 | P41227 | A0A0B2W6H8 |
| PDIA5 | Q14554 | A0A8C0TL18 |
| NMI | Q13287 | A0A8P0NZK0 |
| NCC27 | O00299 | A0A8C0L4R5 |
| COL11A2 | P13942 | A0A8I3PB22 |
| KSYK | P43405 | A0A8C0M271 |
| ARHGP | Q86VW2 | A0A8C0RE44 |

TABLE 2

| | | Uniprot | Uniprot |
|---|---|---|---|
| | | ID | ID |
| Rank | Biomarker | (Human) | (Canine) |
| 1 | CARD9 | Q9H257 | A0A8I3N256 |
| 2 | GDF-11/8 | O95390 | A0A8I3MVU8 |
| | | O14793 | Q6UKZ8 |
| 3 | FGF9 | P31371 | A0A8C0TNC4 |
| 4 | AMGO1 | Q86WK6 | A0A8I3MLU9 |
| 5 | CF6 | P18859 | A0A0B2VY56 |
| 6 | RN215 | Q9Y6U7 | A0A8P0NEJ2 |
| 7 | CEI | Q86SI9 | A0A8C0N5G3 |
| 8 | AGAP2 | Q99490 | A0A8I3NL11 |
| 9 | SDSL | Q96GA7 | A0A8C0PJ89 |
| 10 | RUNX3 | Q13761 | A0A8C0PLB3 |
| 11 | MDGA2 | Q7Z553 | A0A8P0PIE7 |
| 12 | ARHG1 | Q92888 | A0A8C0QQ66 |
| 13 | ATG4B | Q9Y4P1 | A0A8C0PRI8 |
| 14 | Collagen II | P02458 | A0A8C0PFU8 |
| 15 | ADAMTS-5 | Q9UNA0 | A0A8P0SDL0 |
| 16 | CHD7 | Q9P2D1 | A0A8C0SX81 |
| 17 | SEM4D | Q92854 | A0A8C0PK44 |
| 18 | SEPTIN10 | Q9P0V9 | A0A8C0RTA3 |
| 19 | Laminin-2 | P24043 | A0A8I3RRA4A0A8I3PL58 |
| | | P07942 | A0A8C0M627 |
| | | P11047 | |
| 20 | Aurora kinase A | O14965 | A0A8I3S724 |
| 21 | PIK3CA/PIK3R1 | P42336 | A0A5F4C2B1 |
| | | P27986 | A0A8I3N971 |
| 22 | PURA2 | P30520 | A0A8C0P3B8 |
| 23 | WFDC8 | Q8IUA0 | A0A8I3NZM8 |
| 24 | WWOX | Q9NGC7 | A0A8I3P991 |

TABLE 3

| | | Uniprot | Uniprot |
|---|---|---|---|
| | | ID | ID |
| Rank | Biomarker | (Human) | (Canine) |
| 1 | NTRI | Q9P121 | A0A8I3MR44 |
| 2 | ADAMTS-5 | Q9UNA0 | A0A8P0SDL0 |
| 3 | ARHG1 | Q92888 | A0A8C0QQ66 |
| 4 | RUNX3 | Q13761 | A0A8C0PLB3 |
| 5 | CEI | Q86SI9 | A0A8C0N5G3 |
| 6 | AGAP2 | Q99490 | A0A8I3NL11 |
| 7 | NXF1 | Q9UBU9 | A0A8I3N3V9 |
| 8 | IGDC4 | Q8TDY8 | A0A8C0K7I6 |
| 9 | Laminin-2 | P24043 | A0A8I3RRA4A0A8I3PL58 |
| | | P07942 | A0A8C0M627 |
| | | P11047 | |
| 10 | NTRI | Q9P121 | A0A8I3MR44 |
| 11 | RCD1 | Q92600 | A0A0B2UZ33 |
| 12 | CARD9 | Q9H257 | A0A8I3N256 |
| 13 | SRCN1 | P12931 | A0A8I3SBF9 |
| 14 | NMES1 | Q9C002 | A0A8C0N9H4 |
| 15 | sE-Selectin | P16581 | A0A8I3MRV9 |
| 16 | AGO1 | Q9UL18 | A0A8C0RZU0 |
| 17 | Calcineurin | Q08209 | A0A8I3PS73 |
| | | P63098 | A0A8I3PQY6 |
| 18 | GDF-11/8 | O95390 | A0A8I3MVU8 |
| | | O14793 | Q6UKZ8 |
| 19 | STK24 | Q9Y6E0 | A0A8I3S1Q3 |
| 20 | Ezrin | P15311 | A0A8C0NAG2 |
| 21 | Thioredoxin | Q16881 | A0A8C0K7A3 |
| | reductase 1 | | |
| 22 | HSP 70 | P0DMV8 | A0A0B2V5X6 |
| 23 | BOC | Q9BWV1 | A0A8C0TJR0 |
| 24 | CNOT1 | A5YKK6 | A0A8I3MQ47 |
| 25 | MARCKSL1 | P49006 | A0A8C0TGC8 |
| 26 | MK10 | P53779 | A0A8C0Z6V9 |
| 27 | PABP4 | Q13310 | A0A8I3N997 |
| 28 | CSNK1G2 | P78368 | A0A8C0L0H2 |
| 29 | Moesin | P26038 | A0A8C0TC91 |
| 30 | PGCB | Q96GW7 | H6VX53 |
| 31 | TIM50 | Q3ZCQ8 | A0A8I3MU85 |
| 32 | MFAP5 | Q13361 | A0A8C0PCR8 |
| 33 | SYTL4 | Q96C24 | A0A8C0NF93 |
| 34 | SEPTIN10 | Q9P0V9 | A0A8C0RTA3 |
| 35 | PKB gamma | Q9Y243 | A0A8C0NWA6 |
| 36 | PCDH8 | O95206 | A0A8C0L1A7 |
| 37 | CAMK2B | Q13554 | A0A8C0RD52 |
| 38 | MEK1 | Q02750 | A0A8C0NAT7 |
| 39 | HSP 10 | P61604 | A0A8C0KIA5 |
| 40 | OAF | Q86UD1 | A0A8C0M6K0 |
| 41 | MDGA2 | Q7Z553 | A0A8P0PIE7 |
| 42 | BARK1 | P25098 | A0A8I3NE91 |
| 43 | JNK2 | P45984 | A0A8C0QAU7 |
| 44 | LYN | P07948 | A0A8I3PUN0 |
| 45 | NUCB1 | Q02818 | A0A8C0PVG8 |
| 46 | MAPK14 | Q16539 | A0A8C0SYM9 |
| 47 | STAT3 | P40763 | A0A8C0QJT6 |
| 48 | HMHA1 | Q92619 | A0A8C0QDY6 |
| 49 | FGF9 | P31371 | A0A8C0TNC4 |
| 50 | RPAB4 | P53803 | A0A8C0NDJ7 |
| 51 | CAMK2A | Q9UQM7 | A0A8I3NIS8 |
| 52 | LCN8 | Q6JVE9 | A0A8I3MWP0 |
| 53 | RIR2B | Q7LG56 | A0A8C0NXM7 |
| 54 | MP2K4 | P45985 | A0A8I3MIB2 |
| 55 | RHG25 | P42331 | A0A8C0P719 |
| 56 | PP1G | P36873 | Q8MJ46 |
| 57 | CHKB | Q9Y259 | A0A8I3PU66 |
| 58 | MYCBP | Q99417 | A0A8C0M044 |
| 59 | PKB | P31749 | A0A8C0MEP1 |
| 60 | WFDC8 | Q8IUA0 | A0A8I3NZM8 |
| 61 | WWP1 | Q9H0M0 | A0A8C0MK13 |
| 62 | UBS3B | Q8TF42 | A0A8C0LWY3 |
| 63 | CAMK2D | Q13557 | A0A8C0NZM8 |
| 64 | DUSP13 | Q6B8I1 | A0A8C0NVL5 |
| 65 | HSP 90a | P07900 | A0A8I3MR78 |
| 66 | GSK-3 alpha | P49840 | A0A8I3MIZ4 |
| 67 | CSNK1G2 | P78368 | A0A8C0L0H2 |
| 68 | RN215 | Q9Y6U7 | A0A8P0NEJ2 |
| 69 | COMMD7 | Q86VX2 | A0A8C0Q728 |
| 70 | M2-PK | P14618 | A0A8C0YZD9 |
| 71 | AP4M1 | O00189 | A0A8I3RQU5 |
| 72 | MK08 | P45983 | A0A8I3NY64 |
| 73 | HSP70 protein 8 | P11142 | A0A8C0KE17 |
| 74 | Histidyl-tRNA | P12081 | A0A8I3MNZ5 |
| | synthetase | | |
| 75 | TrkB | Q16620 | Q6JDI7 |
| 76 | PIK3CA/PIK3R1 | P42336 | A0A5F4C2B1 |
| | | P27986 | A0A8I3N971 |
| 77 | MESD2 | Q14696 | A0A0B2VNA1 |
| 78 | PGAM2 | P15259 | A0A8C0TSU2 |
| 79 | KBP | Q96EK5 | A0A8I3MUW8 |
| 80 | CSK | P41240 | A0A8C0RH82 |
| 81 | ETS2 | P15036 | A0A8C0RTI9 |
| 82 | OST48 | P39656 | Q05052 |
| 83 | SUMF2 | Q8NBJ7 | A0A8I3NQ68 |
| 84 | USO1 | O60763 | A0A8I3Q5G1 |
| 85 | Angiostatin | P00747 | P80009 |
| 86 | FER | P16591 | Q9TTY2 |
| 87 | NAA10 | P41227 | A0A0B2W6H8 |
| 88 | PDIA5 | Q14554 | A0A8C0TL18 |
| 89 | NMI | Q13287 | A0A8P0NZK0 |
| 90 | SEM4D | Q92854 | A0A8C0PK44 |
| 91 | NCC27 | O00299 | A0A8C0L4R5 |
| 92 | COL11A2 | P13942 | A0A8I3PB22 |
| 93 | KSYK | P43405 | A0A8C0M271 |
| 94 | ARHGP | Q86VW2 | A0A8C0RE44 |

1. A method for determining the biological age of an animal, wherein the method comprises determining the level at least 1 biomarker in the animal, wherein the biomarker is selected from the biomarkers as listed in Table 1.

2. The method according to claim 1, wherein the levels of five or more biomarkers are determined in the animal, preferably wherein at least 2, at least 3, at least 4, at least 5, at least 10, at least 25, at least 50, at least 75 or at least 100 of the biomarkers are selected from the biomarkers as listed in Table 1.

3. The method according to claim 1, wherein at least 1, at least 2, at least 3, or at least 4 biomarkers are selected from the biomarkers listed as biomarkers 1 to 5 in Table 2.

4. The method according to claim 1, wherein at least 10, at least 15 or at least 20 biomarkers are selected from the biomarkers as listed as biomarkers 1 to 24 in Table 2.

5. The method according to claim 1, wherein the method comprises determining the levels of at least the biomarkers listed as biomarkers 1 to 5 in Table 2.

6. The method according to claim 1, wherein the method comprises determining the levels of at least the biomarkers listed as biomarkers 1 to 10 in Table 2.

7. The method according to claim 1, wherein the method comprises determining the levels of at least all the biomarkers listed in Table 2.

8. The method according to claim 1, wherein at least 1, at least 2, at least 3, or at least 4 biomarkers are selected from the biomarkers listed as biomarkers 1 to 5 in Table 3.

9. The method according to claim 1, wherein at least 10, at least 11 or at least 12 biomarkers are selected from the biomarkers listed as biomarkers 1 to 15 in Table 3.

10. The method according to claim 1, wherein at least 20, at least 25, at least 30, at least 35 or at least 40 biomarkers are selected from the biomarkers as listed as biomarkers 1 to 50 in Table 3.

11. The method according to claim 1, wherein the method comprises determining the levels of at least the biomarkers listed as biomarkers 1 to 5 in Table 3.

12. The method according to claim 1, wherein the method comprises determining the levels of at least the biomarkers listed as biomarkers 1 to 50 in Table 3.

13. The method according to claim 1, wherein the method comprises determining the levels of at least all the biomarkers listed in Table 3.

14. The method according to claim 1, wherein the animal is a canine.

15. The method according to claim 1, wherein the determined levels of the five or more biomarkers are the levels of the biomarkers in a sample obtained from the animal.

16. The method according to claim 15, wherein the sample is a tissue sample, a blood sample, a serum sample, or a plasma sample.

17. A method for selecting a dietary, pharmacological or lifestyle regime for an animal, the method comprising:
(i) determining the biological age of the animal by the method according to claim 1,
(ii) selecting a suitable dietary, pharmacological or lifestyle regime for the animal based on the biological age of the animal as determined in step (i).

18. The method according to claim 17, wherein the suitable dietary,
pharmacological or lifestyle regimen is selected based on the comparison of the determined biological age relative to the chronological age of the animal.

19. A method for determining the efficacy of a dietary, pharmacological or lifestyle regime for improving the biological age of an animal, the method comprising:
(i) determining the biological age of the animal by the method according to claim 1, wherein the biological age is determined from a sample obtained from the animal before the dietary, pharmacological or lifestyle regime,
(ii) determining the biological age of the animal by the method according to claim 1, wherein the biological age is determined from a sample obtained from the animal after the dietary, pharmacological or lifestyle regime, and
(iii) determining if there has been a change in the biological age of the animal between the sample obtained from the animal before and after the dietary, pharmacological or lifestyle regime has been applied.
